(19) United States
(12) Patent Application Publication
(10) Pub. No.: US 2003/0119813 A1
(43) Pub. Date: Jun. 26, 2003

(54) Derivatives of pyrimido[6,1-a]isoquinolin-4-one

(76) Inventors: Alexander William Oxford, Royston (GB); David Jack, Wheathampstead (GB)

Correspondence Address: Dike, Bronstein, Roberts & Cushman, Intellectual Property Patent Practice, EDWARDS & ANGELL, LLP, P.O. Box 9169, Boston, MA, 02209, US

(21) Appl. No.: 09/964,266

(22) Filed: Sep. 26, 2001

(51) Int. Cl. A61K 31/553; A61K 31/519; C07D 498/14; C07D 487/02

(52) U.S. Cl. 514/211.12; 514/267; 540/548; 544/252

(57) ABSTRACT

The invention provides compounds or salts thereof of the general formula (I):

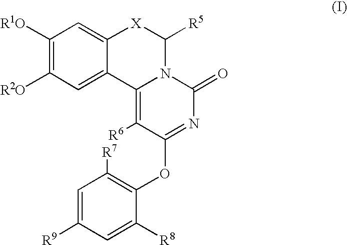

wherein each of R¹and R² independently represents a C₁₋₆ alkyl or C₂₋₇ acyl group; X represents OCH₂ or a group CR³R⁴; wherein each of R³ or R⁴ independently represents a hydrogen atom or a C₁₋₃ alkyl group; R⁵ represents a hydrogen atom or a C₁₋₃ alkyl, C₂₋₃ alkenyl or C₂₋₃ alkynyl group; R⁶ represents a hydrogen atom or a C₁₋₆ alkyl, C₂₋₆ alkenyl, C₂₋₆ alkynyl, amino, C₁₋₆ alkylamino, di(C₁₋₆) alkylamino or C₂₋₇ acylamino group; each of R⁷ and R⁸ independently represents a hydrogen or halogen atom or a hydroxy, trifluoromethyl, C₁₋₆ alkyl, C₂₋₆ alkenyl, C₂₋₆ alkynyl, C₂₋₇ acyl, C₁₋₆ alkylthio, C₁₋₆ alkoxy, C₃₋₆ cycloalkyl; and R⁹ represents a hydrogen or halogen atom or a hydroxy, trifluoromethyl, C₁₋₆ alkyl, C₂₋₆ alkenyl, C₂₋₆ alkynyl, C₂₋₇ acyl, C₁₋₆ alkylthio, C₁₋₆ alkoxy or C₃₋₆ cycloalkyl group. The compounds or salts thereof are useful for treatment of respiratory disorders such as asthma. Compounds of the invention have a longer duration of action than the known compound trequinsin (9,10-dimethoxy-3 methyl-2-mesitylimino-2,3,6,7-tetrahydro-4H-pyrimido[6,1-a]isoquinolin-4-one).

The present invention relates to derivatives of pyrimido[6,1-a]isoquinolin-4-one and their application as inhibitors of phosphodiesterase (PDE) isoenzymes. More particularly the invention relates to 2-phenoxy derivatives of pyrimido[6,1-a]isoquinolin-4-one and their use in medicine for example as bronchodilators with anti-inflammatory properties.

In all cells where cyclic AMP (cAMP) is present as a secondary messenger, intracellular concentrations of cAMP are regulated by the two processes involved in its formation and degradation. Stimulation of membrane bound receptors on the external surface of the cells (e.g. by β-adrenoceptor agonists) results in activation of adenylyl cyclase to generate cAMP from ATP. Phosphodiesterases present in the cell serve to reduce the concentration of cAMP by hydrolysing it to adenosine monophosphate (AMP).

In a disease such as asthma, the principal cells involved in the associated bronchoconstriction and inflammatory processes are subject to inhibitory control by cAMP. Inhibitors of type III phosphodiesterase raise intracellular levels of cAMP, leading to relaxation of bronchial smooth muscle, whereas inhibitors of type IV phosphodiesterase inhibit the release of damaging mediators from pro-inflammatory cells. Thus, in principle, a combined PDE III/IV inhibitor should have the desirable effects of a β-adrenoceptor agonist plus an inhaled anti-inflammatory steroid which are currently the mainstay of treatment in severe asthma. Moreover, a combined PDE III/IV inhibitor given by inhalation should achieve beneficial effects similar to a β-agonist plus inhaled steroid and should be an unusually effective treatment of asthma and other respiratory disorders without the undesirable glucocorticoid effects of the steroid such as osteoporosis and the stunting of growth.

The potential adverse effects of a PDE III/IV inhibitor (e.g. nausea and vomiting, gastric acid secretion, cardiovascular effects such as increased cardiac contractility, vasodilation and potential arrhythmogenic activity) should be avoidable with a compound that is delivered directly to the lungs by inhalation. It is desirable that the substance is long acting, non irritant and has a taste which is not so unpleasant as to have any adverse effect on patient compliance.

An example of a pyrimido[6,1-a]isoquinolin-4-one derivative with PDE III/IV inhibitory activity and known to-possess antihypertensive vasodilator activity is trequinsin (9,10-dimethoxy-3-methyl-2-mesitylimino-2,3,6,7-tetrahydro-4H-pyrimido[6,1-a]isoquinolin-4-one), which is described by De Souza et al., J. Med. Chem. 27 1470-1480 (1984) and in GB-A-1597717.

As described by De Souza et al. and in GB-A-1597717, trequinsin has valuable pharmacological properties, and can be administered to human subjects suffering from, for example, respiratory disorders. However, it is unsuitable for administration by inhalation because of its bitter taste and in vitro data indicate its persistence of action is less than desirable.

It has now been found that it is possible to design certain pyrimido[6,1-a]isoquinolin-4-one derivatives which are PDE inhibitors, which have a longer duration of action relative to trequinsin and other useful properties, such as improved taste.

According to a first aspect of the present invention there is provided a compound of general formula I:

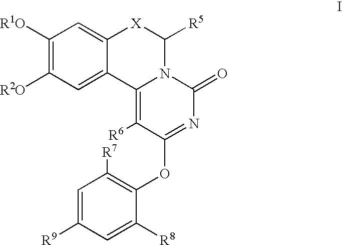

wherein

each of R¹ and R² independently represents a C₁₋₆ alkyl or C₂₋₇ acyl group;

X represents OCH₂ or a group CR³R⁴, wherein each of R³ and R⁴ independently represents a hydrogen atom or a C₁₋₃ alkyl group;

R⁵ represents a hydrogen atom or a C₁₋₃ alkyl, C₂₋₃ alkenyl or C₂₋₃ alkynyl group;

R⁶ represents a hydrogen atom or a C₁₋₆ alkyl, C₂₋₆ alkenyl, C₂₋₆ alkynyl, amino, C₁₋₆ alkylamino, di(C₁₋₆) alkylamino or C₂₋₇ acylamino group;

each of R⁷ and R⁸ independently represents a hydrogen or halogen atom or a hydroxy, trifluoromethyl, C₁₋₆ alkyl, C₂₋₆ alkenyl, C₂₋₆ alkynyl, C₂₋₇ acyl, C₁₋₆ alkylthio, C₁₋₆ alkoxy or C₃₋₆ cycloalkyl group;

R⁹ represents a hydrogen or halogen atom or a hydroxy, trifluoromethyl, C₁₋₆ alkyl, C₂₋₆ alkenyl, C₂₋₆ alkynyl, C₂₋₇ acyl, C₁₋₆ alkylthio, C₁₋₆ alkoxy or C₃₋₆ cycloalkyl group;

or a salt thereof;

excluding the compound 6,7-dihydro-2-phenoxy-9,10-dimethoxy-4H-pyrimido[6,1a]isoquinolin-4-one.

The compound 6,7-dihydro-2-phenoxy-9,10-dimethoxy-4H-pyrimido[6,1a]isoquinolin-4-one, which is excluded from the scope of the first aspect of the present invention, is compound “8f” in De Souza et al, cited above. 6,7-dihydro-2-phenoxy-9,10-dimethoxy-4H-pyrimido[6,1a]isoquinolin-4-one is described in De Souza et al only as being a poor hypotensive agent and is not described as having bronchodilatory activity.

As used herein the term “halogen” or its abbreviation “halo” means fluoro, chloro, bromo or iodo.

As used herein the term “C₁₋₆ alkyl” refers to straight chain or branched chain alkyl groups having from one to six carbon atoms. Illustrative of such alkyl groups are methyl, ethyl, propyl, isopropyl, butyl, isobutyl, sec-butyl, tert-butyl, pentyl, neopentyl and hexyl. C₁₋₄ alkyl groups are preferred.

As used herein the term “C₂₋₃ alkenyl” refers to straight chain or branched chain hydrocarbon groups having from two to three carbon atoms and having in addition one double bond, of either E or Z stereochemistry where applicable. This term would include for example, vinyl and 1-propenyl.

As used herein the term “C₂₋₃ alkynyl” refers to straight chain hydrocarbon groups having from two to three carbon atoms and having in addition one triple bond. This term would include for example, ethynyl and 1-propynyl.

As used herein the term “C₂₋₆ alkenyl” refers to straight chain or branched chain hydrocarbon groups having from two to six carbon atoms and having in addition one double bond, of either E or Z stereochemistry where applicable. This term would include for example, vinyl, 1-propenyl, 1- and 2-butenyl and 2-methyl-2-propenyl. C₂₋₃ alkenyl groups are preferred.

As used herein the term “C₂₋₆ alkyl” refers to straight chain or branched chain hydrocarbon groups having from two to six carbon atoms and having in addition one triple bond. This term would include for example, ethynyl, 1-propynyl, 1- and 2-butynyl, 2-methyl-2-propynyl, 2-pentanyl, 3-pentanyl, 4-pentanyl, 2-hexanyl, 3-hexanyl, 4-hexanyl and 5-hexanyl. C₂₋₃ alkynyl groups are preferred.

As used herein the term “C₁₋₆ alkoxy” refers to straight chain or branched chain alkoxy groups having from one to six carbon atoms. Illustrative of such alkoxy groups are methoxy, ethoxy, propoxy, isopropoxy, butoxy, isobutoxy, sec-butoxy, tert-butoxy, pentoxy, neopentoxy and hexoxy. C₁₋₄ alkoxy groups are preferred.

As used herein the term “C₂₋₇ acyl” refers to straight chain or branched chain acyl groups having from two to seven carbon atoms. Illustrative of such acyl groups are acetyl, propionyl (or propiono or propanoyl), isopropionyl (or isopropiono or isopropanoyl), butyryl (or butanoyl), isobutyryl (or isobutanoyl), pentanoyl (or valeryl), hexanoyl (or capronyl) and heptanoyl.

As used herein the term “C₂₋₇ acyloxy” refers to straight chain or branched chain acyloxy groups having from two to seven carbon atoms. Illustrative of such acyloxy groups are acetyloxy, propionyl (or propiono or propanoyl)oxy, isopropionyl (or isopropiono or isopropanoyl)oxy, butyryl (or butanoyl)oxy, isobutyryl (or isobutanoyl)oxy, pentanoyl (or valeryl)oxy, hexanoyl (or capronyl)oxy and heptanoyloxy. C₂₋₄ acyloxy groups are preferred.

As used herein the term “C₃₋₆ cycloalkyl” refers to an alicyclic group having from three to six carbon atoms. Illustrative of such cycloalkyl groups are cyclopropyl, cyclobutyl, cyclopentyl and cyclohexyl. Cyclopentyl and cyclohexyl groups are preferred.

As used herein the term “C₁₋₆ alkylthio” refers to straight chain or branched chain alkylthio groups having from one to six carbon atoms. Illustrative of such alkylthio groups are methylthio, ethylthio, propylthio, isopropylthio, butylthio, isobutylthio, sec-butylthio, tert-butylthio, pentylthio, neopentylthio and hexylthio. C₁₋₄ alkylthio groups are preferred.

As used herein the term “C₁₋₆ alkylamino” refers to straight chain or branched chain alkylamino groups having from one to six carbon atoms. Illustrative of such alkylamino groups are methylamino, ethylamino, propylamino, isopropylamino, butylamino, isobutylamino, sec-butylamino, tert-butylamino, pentylamino, neopentylamino and hexylamino. CoI alkylamino groups are preferred.

As used herein, the term “di(C₁₋₆) alkylamino” refers to straight chain or branched chain di-alkylamino groups having from one to six carbon atoms in each of the alkyl groups. Illustrative of such dialkylamino groups are di-methylamino, di-ethylamino, di-propylamino, di-isopropylamino, di-butylamino, di-isobutylamino, di-sec-butylamino, di-tert-butylamino, di-pentylamino, di-neopentylamino and di-hexylamino. Di(C₁₋₄)alkylamino groups are preferred.

As used herein, the term “C₂₋₇ acylamino” refers to straight chain or branched chain acylamino groups having from two to seven carbon atoms. Illustrative of such acylamino groups are acetylamino, propionyl (or propiono or propanoyl)amino, isopropionyl (or isopropiono or isopropanoyl)amino, butyryl (or butanoyl)amino, isobutyryl (or isobutanoyl)amino, pentanoyl (or valeryl)amino, hexanoyl (or capronyl)amino and heptanoylamino. C₂₋₄ acylamino groups are preferred.

Where there is a substituent which renders a compound basic, for example when R⁶ is an amino, alkylamino or dialkylamino group, addition of an acid results in a salt. The acid may be any suitable acid, and can be organic or inorganic.

Preferred compounds of general formula I include those in which, independently or in any compatible combination:

each of R¹and R² represents a C₁₋₆ alkyl, preferably a C₁₋₄ alkyl, group;

R¹and R² are the same as each other;

each of R¹and R⁴ represents a hydrogen atom;

R⁵ represents a hydrogen atom;

R⁶ represents a hydrogen atom;

each of R⁷ and R⁸ represents a C₁₋₆ alkyl, preferably methyl, ethyl or isopropyl, group;

R⁷ and R⁸ are the same as each other;

R⁹ represents a halogen atom or a methyl or acetyl group.

Exemplary compounds include:

1. 6,7-Dihydro-2-(2,6-dimethylphenoxy)-9,10-dimethoxy-4H-pyrimido-[6,1-a]iso-quinolin-4-one;

2. 2-(2,6-dimethylphenoxy)-6,7-dihydro-9,10-dimethoxy-4H-pyrimido-[6,1-a]iso-quinolin-4-one;

3. 6,7-Dihydro-2-(2,6-diisopropylphenoxy)-9,10-dimethoxy-4H-pyrimido-[6,1-a]isoquinolin-4-one;

4. 6,7-Dihydro-9,10-dimethoxy-2-(2,4,6-trimethylphenoxy)-4H-pyrimido-[6,1-a]isoquinolin-4-one;

5. 2-(4-Chloro-2,6-dimethylphenoxy)-6,7-dihydro-9,10-dimethoxy-4H-pyrimido-[6,1-a]isoquinolin-4one; 6. 2-(4Bromo-2,6-dimethylphenoxy)-6,7-dihydro-9,10-dimethoxy-4H-pyrimido-[6,1-a]isoquinolin-4-one;

7. 6,7-Dihydro-9,10-dimethoxy-2-(4-ethanoyl-2,6-dimethylphenoxy)-4H-pyrimido-[6,1-a]isoquinolin-4-one;

8. 2-(2,6-Dichlorophenoxy)-6,7-dihydro-9,10-dimethoxy-4H-pyrimido-[6,1-a]isoquinolin-4-one;

9. 9,10-Dimethoxy-2-(2-methylphenoxy)-6,7-dihydro-4H-pyrimido[6,1-a]isoquinolin-4-one;

10. 9,10-Dimethoxy-2-(2-isobutylphenoxy)-6,7-dihydro-4H-pyrimido[6,1-a]isoquinolin-4one;

11. 2-(2-tert-butylphenoxy)-9,10-dimethoxy-6,7-dihydro-4H-pyrimido[6,1-a]isoquinolin-4-one;

12. 9,10-Dimethoxy-2-(2-ethylphenoxy)-6,7-dihydro-4H-pyrimido[6,1-a]isoquinolin-4-one;

13. 2-(2-Cyclopentylphenoxy)-9,10-dimethoxy-6,7-dihydro-4H-pyrimido[6,1-a]isoquinolin-4one.

The compounds 2-(2,6-dimethylphenoxy)-6,7-dihydro-9,10-dimethoxy-4H-pyrimido-[6,1-a]isoquinolin-4-one and 6,7-Dihydro-2-(2,6-diisopropylphenoxy)-9,10-dimethoxy-4H-pyrimido-[6,1-a]isoquinolin-4-one are particularly preferred.

Compounds of general formula I may be prepared by any suitable method known in the art and/or by the following process, which itself forms part of the invention.

According to a second aspect of the invention, there is provided a process for preparing a compound of general formula I as defined above, excluding the compound 6,7-dihydro-2-phenoxy-9,10-dimethoxy-4H-pyrimido[6,1a]isoquinolin-4one, the process comprising:

(a) reacting a compound of general formula II:

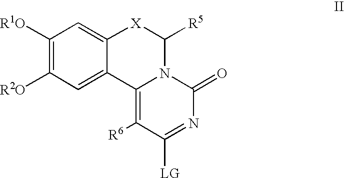

wherein R¹, R², R⁵, R⁶ and X are as defined for general formula I and LG represents a leaving group, with a compound of general formula III

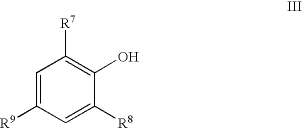

wherein R⁷, R⁸ and R⁹ are as defined for general formula I; or

(b) when X in general formula I represents a group CR³R⁴, wherein R³ represents a hydrogen atom, R⁴ represents a hydrogen atom or a C₁₋₃ alkyl group, and R⁵ represents a hydrogen atom or a C₁₋₃ alkyl group, hydrogenating a compound of general formula IX:

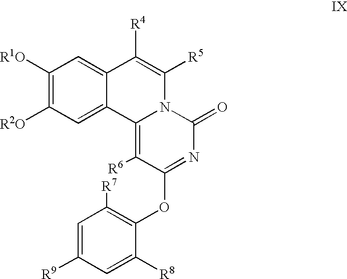

wherein R¹, R², R⁷, R⁸ and R⁹ are as defined for general formula 1; and

(c) optionally converting a compound of general formula I so formed into another compound of general formula I.

In compounds of general formula II, the leaving group LG may be chlorine, a thioalkyl group, preferably thiomethyl, or an alkylsulphonyl group, preferably methylsulphonyl. Preferably it is chlorine.

The reaction conditions of step (a) are generally such as to favour the reaction, which is a nucleophilic displacement which is preferably carried out in a suitable solvent such as dimethylformamide or isopropanol in the presence of a base such as potassium carbonate. Suitable reaction conditions may be found in GB-A-1597717 and EP-A-0124893, which disclose the preparation of related compounds.

The reaction of step (a) is generally applicable for producing compounds of general formula I where R⁶ represents a hydrogen atom or a C₁₋₆ alkyl, C₂₋₆ alkenyl, C₂₋₆ alkynyl, amino, C₁₋₆ alkylamino or C₂₋₇ acylamino group and R¹ to R⁵ and R⁷ to R⁹ and X have the meanings given above.

Compounds of general formula II where LG represents a chlorine atom may be prepared by reacting a compound of general formula IV or a compound of general formula V with phosphorous oxychloride, or by heating a compound of general formula IV with phosphorous pentachloride:

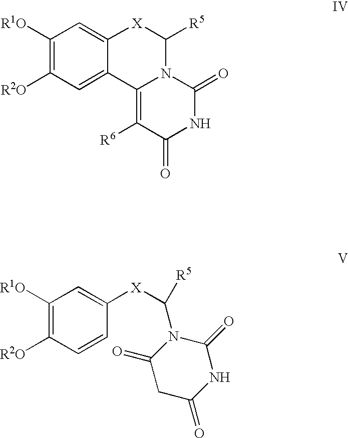

wherein R¹, R², R⁵, R⁶ and X are as defined for general formula I. Compounds of general formula II where LG represents a thioalkyl group may be prepared from compounds of general formula IV by heating with phosphorous pentasulphide in a solvent such as dioxan or pyridine to give initially the intermediate thio derivative of compound of formula IV which, on treatment with an alkylating agent such as an alkyl iodide eg. methyl iodide, in a suitable solvent such as tetrahydrofuran or ethyl acetate, gives the thioalkyl compound. Oxidation of the thioalkyl compound with, for example, 3-chloroperbenzoic acid in a solvent such as methylene chloride, gives the alkylsulphone derivative.

Compounds of general formula III are substituted phenols which are either known in the art and available from commercial sources or may readily be prepared by methods known per se, for example from the correspondingly substituted aniline compound via hydrolysis of the diazonium salt.

Compounds of general formula IV may be prepared by reacting a compound of general formula V, wherein R¹, R², R⁵ and R⁶ are as defined for general formula I, with a cyclodehydrating agent such as phosphorous oxychloride, under less vigorous condition, ie lower temperatures, than those required to give compounds of the general formula II where LG represents a chlorine atom. An alternative method has been described in NL-A-6,401,827 (Hoffmann-La Roche) which involves reacting the carbanoylmethylene-tetrahydroisoquinoline, formula VIII, with diethyl carbonate in ethanolic sodium ethoxide:

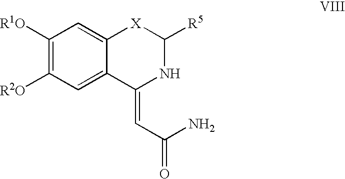

Compounds of general formula V may be prepared by reacting a compound of general formula VI

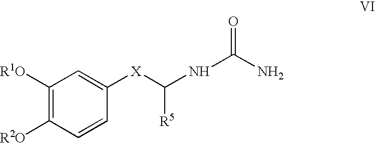

wherein R¹, R², R⁵ and X are as defined for general formula I with R⁶CH(CO₂Et)₂, wherein R⁶ is as defined for general formula I, and a strong base such as sodium ethoxide in hot ethanolic solution. Alternatively, the corresponding dimethyl ester can be employed in the presence of hot methanolic sodium methoxide.

Compounds of general formula VI may be prepared by reacting a compound of general formula VII

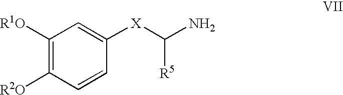

wherein R¹, R², R⁵ and X are as defined for general formula I, with urea by heating at 160,° C. Alternatively, compounds of formula VII may be reacted with potassium cyanate in the presence of acetic acid in a suitable solvent such as ethanol.

Compounds of general formula VII are either known in the art or may readily be prepared by methods known per se. For example, the preparation of 1-(3,4-dimethoxyphenethyl)barbituric acid has been described by B. Lal et al. J. Med. Chem. 27 1470-1480 (1984).

Turning now to step (b), the reaction conditions of step (b) are generally to favour the hydrogenation reaction, and the reaction is generally carried out in a suitable solvent such as an alcohol, eg ethanol, with a noble metal catalyst such as palladium, platinum, rhodium or nickel, at room temperature. The catalyst may be supported, for example on charcoal or alumina.

Compounds of general formula IX may be prepared from a compound of general formula X:

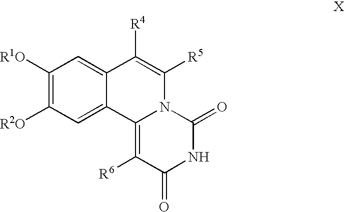

wherein R¹, R² and R⁶ are as defined for general formula I, and R⁴ and R⁵ independently represent a hydrogen atom or a C₁₋₃ alkyl group. The reactions are conducted as described above for converting a compound of general formula IV to a compound of general formula I via compounds of general formula II and the preferred reaction conditions correspond accordingly.

Compounds of general formula X may be prepared from compounds of general formula IV (wherein X represents a group CR³R⁴, wherein R³ represents a hydrogen atom, R⁴ represents a hydrogen atom or a C₁₋₃ alkyl group; and R⁵ represents a hydrogen atom or a C₁₋₃ alkyl group) by heating, with a noble metal catalyst such as palladium, platinum, rhodium or nickel at a temperature of 300 to 350° C. The catalyst may be supported on charcoal or alumina and the reaction carried out in an inert solvent such as an aromatic hydrocarbon, eg p-cymene.

In optional step (c), a compound of general formula I may be converted into another compound of general formula I. For example, compounds of general formula I where R⁶ represents NH₂ may be converted into compounds of general formula I where R⁶ represents a C₁₋₆ alkylamino group by standard chemistry, such as by alkylation of a protected derivative such as an acyl or a p-toluenesulphonyl derivative followed by removal of the protecting group, such as by acid hydrolysis. Compounds of general formula I where R⁶ represents a di(C₁₋₆) alkylamino group may be prepared by direct alkylation of the alkylamino derivative. Compounds of general formula I wherein R⁵, R⁶, R⁷, R⁸ and/or R⁹ represent a C₂₋₃ alkenyl, C₂₋₆ alkenyl, C₂₋₃ alkynyl or C₂₋₆ alkynyl group may be hydrogenated to give the corresponding compound with saturated bonds. The reaction conditions for the hydrogenation are as outlined above for step b).

According to a third aspect, the present invention provides a composition comprising a compound of general formula I, including the excluded compound (6,7-dihydro-2-phenoxy-9,10-dimethoxy-4H-pyrimido[6,1a]isoquinolin-4-one), and a veterinarily or pharmaceutically acceptable carrier or diluent. Preferably the composition is a pharmaceutical composition for human medicine.

Compounds of the present invention are PDE inhibitors and thus possess valuable pharmacological properties, such as bronchodilator activity as demonstrated by the inhibition of field-stimulated contraction of guinea-pig isolated trachea, and anti-inflammatory activity as illustrated in studies on human mononuclear cells stimulated by PHA (phytohaemagglutinin). In vitro and in vivo data indicate the compounds have a long duration of action, as demonstrated by their persistent protective effects against histamine induced bronchospasm in the guinea-pig when inhaled directly into the lungs as a dry powder. The invention therefore also relates to acute, chronic or prophylactic treatment of patients suffering from respiratory disorders including, in particular, asthma, allergic asthma, hay fever, allergic rhinitis, bronchitis, chronic obstructive pulmonary disease (COPD), adult respiratory distress syndrome (ARDS), and cystic fibrosis. They may also be used topically in skin disorders such as atopic dermatitis and psoriasis, or in ocular inflammation or any other disease including cerebral ischaemia or auto-immune diseases in which increasing intracellular concentrations of cAMP is considered beneficial.

One or more compounds as set out in the first aspect of the invention may be present in association with one or more non-toxic pharmaceutically and/or veterinarily acceptable carriers and/or diluents and/or adjuvants and/or propellants and, if desired, other active ingredients. Suitable carriers or diluents are known in the art (eg Handbook of Pharmaceutical Excipients (1994) 2nd Edition, Eds. A. Wade/P J Weller, The Pharmaceutical Press, American Pharmaceutical Association).

Preferably, the compounds and the compositions of the present invention are administered by inhalation, for example by aerosols or sprays which can disperse the pharmacological active ingredient in the form of a powder or in the form of a solution or suspension. Pharmaceutical compositions with powder-dispersing properties usually contain, in addition to the active ingredient, a liquid propellant with a boiling point below room temperature and, if desired, adjuncts, such as liquid or solid non-ionic or anionic surfactants and/or wetting agent to form a stable dispersion. Pharmaceutical compositions in which the pharmacological active ingredient is in solution contain, in addition to this, a suitable propellant, and furthermore, if necessary, an additional solvent and/or a stabiliser. Instead of the propellant, compressed air can also be use, it being possible for this to be produced as required by means of a suitable compression and expansion device. Pharmaceutical compositions may also be delivered by breath activated inhalation devices. Dry powder compositions are preferred for administration by inhalation.

According to a fourth aspect, the present invention provides a compound of general formula I or a composition containing a compound of general formula I, including the excluded compound (6,7-dihydro-2-phenoxy-9,10-dimethoxy-4H-pyrimido[6,1a]isoquinolin-4-one), for use in medicine.

Compounds of the present invention are useful as inhibitors of phosphodiesterase isoenzymes. The compounds or compositions of the present invention may be used to prevent or treat any disease in which the compounds or compositions are useful, but particularly a disease in which raising the intracellular concentration of cAMP is desirable. Examples of diseases against which compounds are useful include respiratory disorders including, in particular, asthma, bronchitis, chronic obstructive pulmonary disease (COPD), adult respiratory distress syndrome (ARDS), allergic asthma, hay fever, allergic rhinitis, and cystic fibrosis. They may also be used topically in skin disorders such as atopic dermatitis or psoriasis, ocular inflammation, or any other disease including cerebral ischaemia or auto-immune diseases in which increasing intracellular concentrations of cAMP is considered beneficial.

This aspect of the invention is particularly relevant to the treatment of humans, but is also applicable to general veterinary industry, in particular domestic animals such as dogs and cats and farm animals such as horses, pigs, cattle, sheep, etc.

Dosage levels of the order of about 0.02 mg to about 200 mg, to be taken up to three times daily, are useful in the treatment of the above-mentioned conditions. More particularly, a dosage range of about 0.2 mg to about 20 mg, taken up to three times daily, is effective. The particular dosage regime will however ultimately be determined by the attending physician and will take into consideration such factors as the medication being used, age, weight, severity of symptoms and/or severity of treatment being or to be applied, method of administration of the medication, adverse reactions and/or other contraindications.

The medication according to this aspect of the invention may be given to a patient together with other active agents, which may for example be a different compound of the present invention, or other compounds. Examples include β₂-adrenoceptor agonists, glucocorticoid steroids, xanthine derivatives, antihistamine compounds, leukotriene antagonists, inhibitors of leukotriene synthesis and/or combinations thereof.

According to a fifth aspect, the present invention provides the use of a compound of general formula I, including the excluded compound (6,7-dihydro-2-phenoxy-9,10-dimethoxy-4H-pyrimido[6,1a]isoquinolin-4-one), in the manufacture of an inhibitor of a type III/IV phosphodiesterase isoenzyme. The invention encompasses the use of a compound of general formula I, including the excluded compound (6,7-dihydro-2-phenoxy-9,10-dimethoxy-4H-pyrimido[6,1a]isoquinolin-4-one), in the manufacture of a bronchodilator and/or an anti-asthmatic medication and/or a medicament for the prevention or treatment of chronic obstructive pulmonary disease (COPD).

The invention also relates to a method for the treatment or prevention of a disease in a mammal where a phosphodiesterase isoenzyme inhibitor and/or a bronchodilator would be expected to be of benefit, which method comprises administering to said mammal an amount of an effective, non-toxic amount of a compound of general formula I, including the excluded compound (6,7-dihydro-2-phenoxy-9,10-dimethoxy-4H-pyrimido[6,1a]isoquinolin-4-one). The invention encompasses a method of treating or preventing asthma and/or chronic obstructive pulmonary disease (COPD) in a mammal.

Preferred features of each aspect of the invention apply to each other aspect of the invention, mutatis mutandis.

The following examples, without being limiting, illustrate the invention, with reference to the following figures:

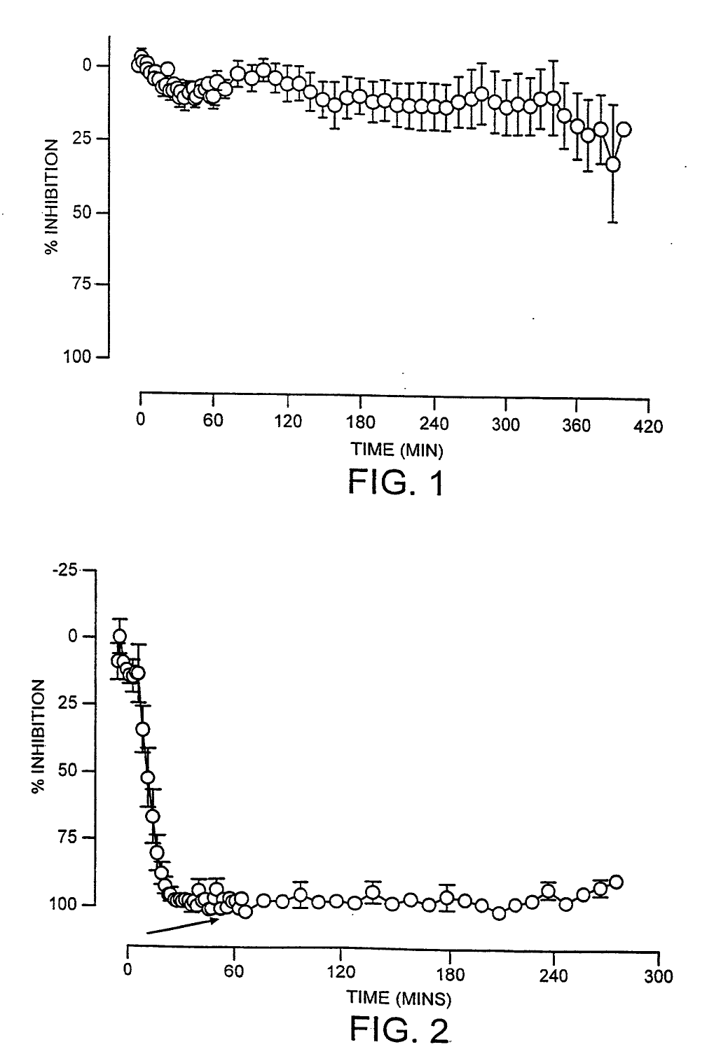

FIG. 1, referred to in Example A(i) below, is a graph showing the effect of DMSO (0.05%) on cholinergic contractile response in superfused guinea pig trachea number of experiments (n) is 10);

FIG. 2, referred to in Example A(i) below, is a graph showing the effect of 10 μM of the compound of Example 3 on the contraction of guinea pig trachea to electrical field stimulation over time (number of experiments (n) is 3), wherein the arrow denotes commencement of washout period;

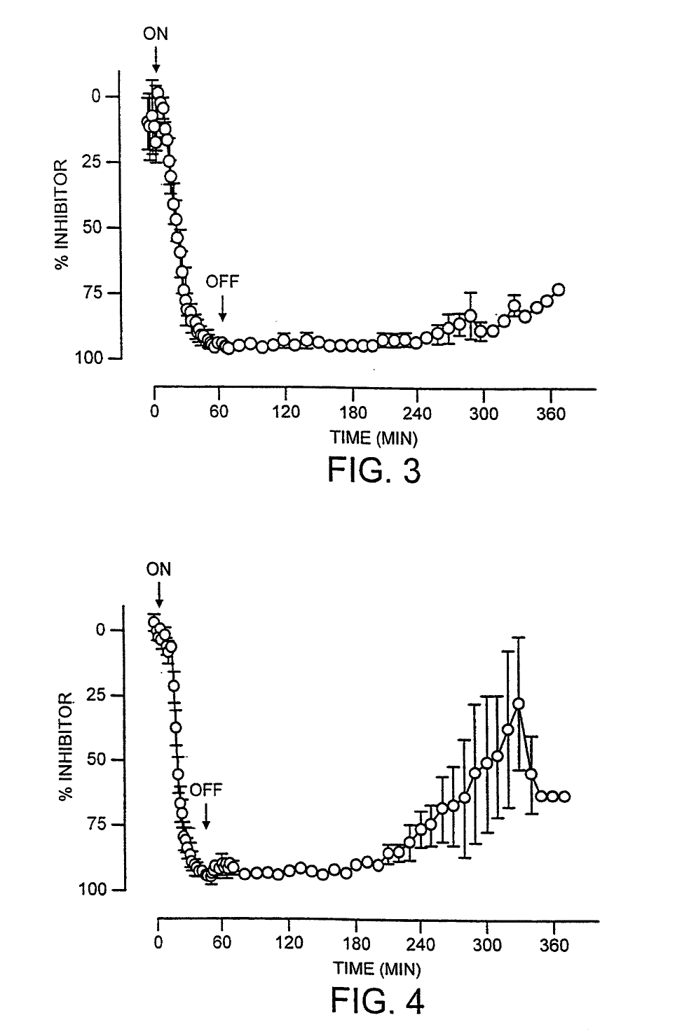

FIG. 3, referred to in Example A(i) below, is a graph showing the effect of 10 μM of the compound of Example 11 on cholinergic contractile response in superfused guinea-pig trachea (number of experiments (n) is 3), wherein the arrow denotes commencement of washout period;

FIG. 4, referred to in Example A(i) below, is a graph showing the effect of 10 μM of the compound of Example 12 on cholinergic contractile response in superfused guinea-pig trachea (number of experiments (n) is 3), wherein the arrow denotes commencement of washout period;

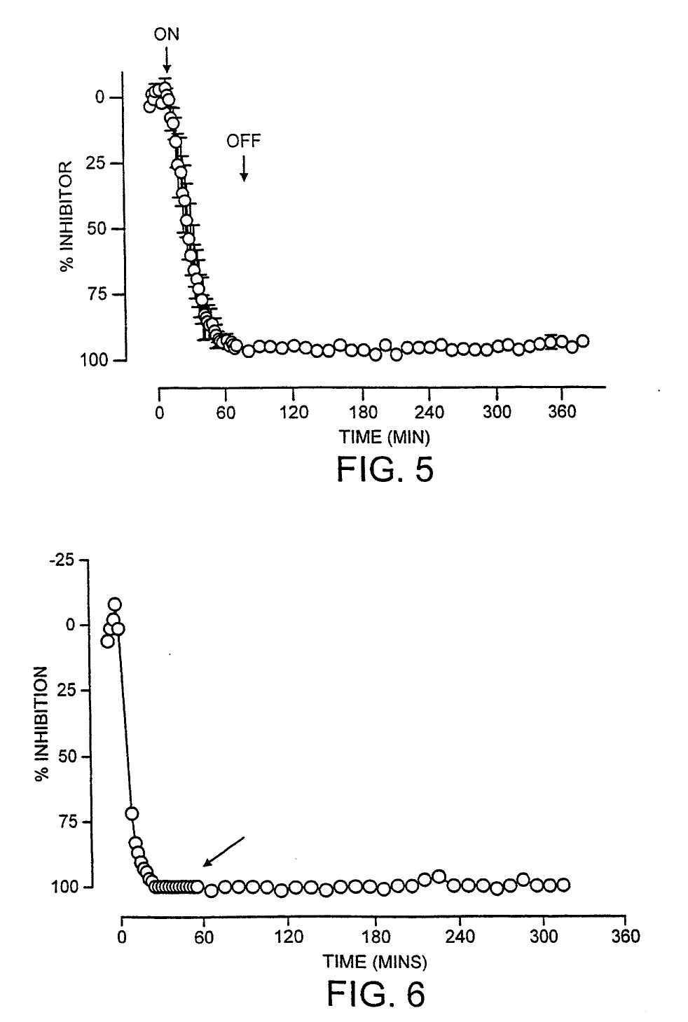

FIG. 5, referred to in Example A(i) below, is a graph showing the effect of 10 μM of the compound of Example 13 on cholinergic contractile response in superfused guinea-pig trachea (number of experiments (n) is 3), wherein the arrow denotes commencement of washout period;

FIG. 6, referred to in Example A(ii) below, is a graph showing the effect of 10 μM of the compound of Example 1 on the contraction of guinea pig trachea to electrical field stimulation over time (n=1), wherein the arrow denotes commencement of washout period;

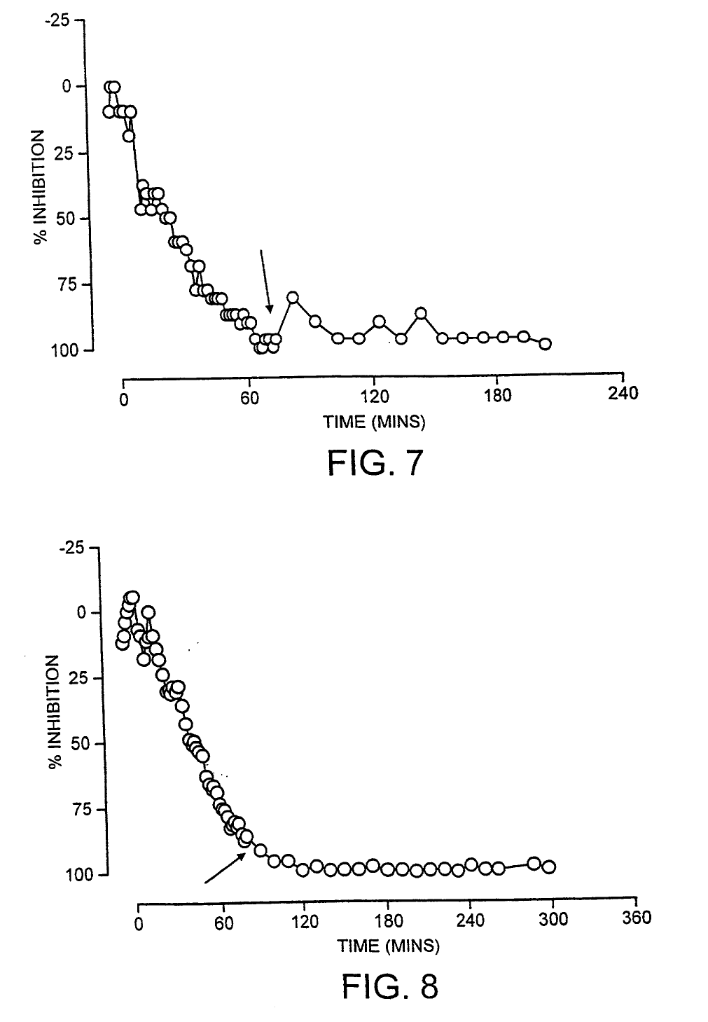

FIG. 7, referred to in Example A(ii) below, is a graph showing the effect of 10 μM of the compound of Example 8 on the contraction of guinea pig trachea to electrical field stimulation over time (n=1), wherein the arrow denotes commencement of washout period;

FIG. 8, referred to in Example A(ii) below, is a graph showing the effect of 10 μM of the compound of Example 2 on contraction of guinea pig trachea to electrical field stimulation over time (n—I), wherein the arrow denotes commencement of washout period;

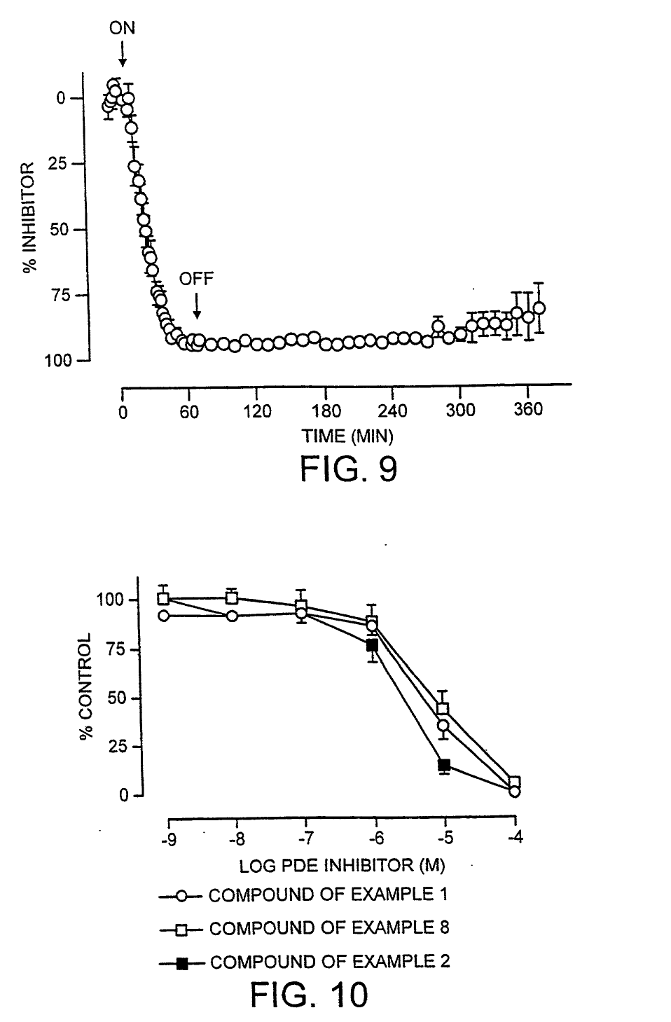

FIG. 9 referred to in Example A(i) below, is a graph showing the effect of 10 μM of the compound of Example 10 on contraction of guinea pig trachea to electrical field stimulation;

FIG. 10, referred to in Example B below, is a graph showing the effect of various compounds of the present invention against proliferation of human mononuclear cells stimulated by PHA, wherein each point represents the mean of six experiments, and vertical lines represent standard error of the mean.

### PREPARATION 1

### Synthesis of 2-Chloro-6,7-dihydro-9,10-Dimethoxy-4H-pyrimido-[6,1-a]isoquinolin-4-one

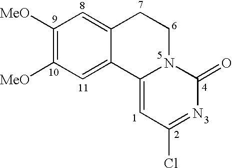

A mixture of 1-(3,4-dimethoxyphenyl) barbituric acid (70 g, 0.24 mol), prepared according to the method described in B. Lal et al. J. Med. Chem. 27 1470-1480 (1984), and phosphorus oxychloride (300 ml, 3.22 mol) was refluxed for 2.5 h. The excess phosphorous oxychloride was removed by distillation (20 mmHg) on warming. After cooling the residue was slurried in dioxan (100 ml) and cautiously added to a vigorously stirred ice/water solution (11). Chloroform (11) was added and the resulting mixture was basified with 30% sodium hydroxide solution. The organic layer was separated and the aqueous phase further extracted with chloroform (2×750 ml). The combined organic extracts were washed with water (1.51), dried over magnesium sulphate and concentrated in vacuo to leave a gummy material (90 g). This was stirred in methanol for a few minutes, filtered and washed with methanol (200 ml), diethyl ether (2×200 ml) and dried in vacuo at 40° C. to yield the title compound as a yellow/orange solid. 47 g, 62%

(300 MHz, CDCl₃) δ 2.96 (2H, t, C(₇) H2); 3.96 (6H, s, 2×OCH₃; 4.20 (2H, t, C₍₆₎H₂); 6.61 (1H, s, C₍₁₎H); 6.76 (1H, s, Ar—H); 7.10(1H, s, Ar—H).

### PREPARATION 2

### 2,6-Diethylphenol

To a solution of 2,6-diethylaniline (14.9 g, 0.10 mol) in glacial acetic acid (200 ml) was added dropwise concentrated sulphuric acid (90 ml) with stirring and cooling to maintain temperature between 10-20° C. The solution was then stirred at 0° C. while sodium nitrite (9.0;, 0.13 mol) in water (50 ml) was added dropwise at such a rate as to keep the temperature below 5° C. The mixture was stirred for a further 20 minutes at 0° C. then an ice cold solution of urea (3.0 g) in water (300 ml) was added slowly to produce an amber solution. A mixture of concentrated sulphuric acid (150 ml) and water (600 ml) was stirred and heated to gentle reflux while the ice cold solution of the diazonium salt was added dropwise maintaining a gentle reflux. It is important that the diazonium salt solution drops directly into the sulphuric acid solution without touching the sides of the flask. After the addition the mixture was stirred for a further 10 minutes at reflux then for 18 h at room temperature. The dark red brown product was removed by filtration, washed with water and dissolved in dichloromethane. The solution was dried over magnesium sulphate and evaporated to obtain a solid which was purified by column chromatography (Silica gel; 2:1 petroleum ether 40-60° C. dichloromethane) to give the title compound as colourless crystals. 9.6 g, 64%

mpt.: 38-39° C.

(60 MHz, CDCl₃) δ 1.23 (6H, t, CH₃); 2.63 (4H, q, CH₂); 4.67 (1H, s, OH); 6.90-7.20 (3H, m, Ar—H).

### EXAMPLE 1

### Synthesis of 6,7-Dihydro-2-(2,6-dimethylphenoxy)-9,10-dimethoxy-4H-pyrimido-[6,1-a]isoquinolin-4-one

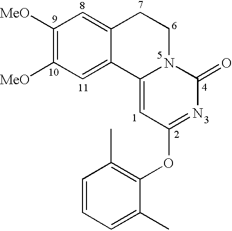

2-Chloro-6,7-dihydro-9,10-dimethoxy-4H-pyrimido-[6,1-a]isoquinolin-4-one (20 g, 68.4 mmol), prepared according to Preparation 1, and 2,6-dimethylphenol (obtained from Aldrich) (25.1 g, 167 mmol) were dissolved in anhydrous N,N-dimethyl formamide (160 ml). Potassium carbonate (28.3 g, 205 mmol) was added and the mixture heated to 90° C. under nitrogen for 3 h. The mixture was then cooled, added to water (600 ml) and extracted with ethyl acetate (3×300 ml). The combined organic extracts were washed with sodium bicarbonate (300 ml), water (2×500 ml) and brine (300 ml), dried over magnesium sulphate and concentrated in vacuo to give a gummy residue. This was triturated with ether (250 ml), isolated by filtration and re-crystallised from methanol (300 ml) to give the title compound as a pale yellow solid. 20 g, 67%

mpt.: 216-218° C.

(300 MHz, d₆DMSO) δ 2.05(6H, s, 2×CH₃); 2.95 (2H, t, C₍₇₎H₂); 3.85 (3H, s, OCH₃); 3.90(3H, s, OCH₃); 4.05 (2H, t, C₍₆₎H₂); 6.95-7.15 (5H, m, Ar—H); 7.55 (1H, s, Ar—H).

### EXAMPLE 2

### Synthesis of 6,7-Dihydro-2-(2,6-diisopropylphenoxy-4-9,10-dimethoxy-4H-pyrimido-[6,1-a]isoquinolin-4-one

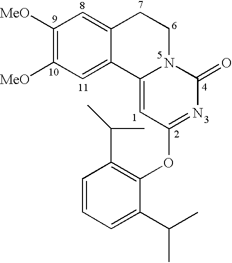

2-Chloro-6,7-dihydro-9,10-dimethoxy-4H-pyrimido-[6,1-a]isoquinolin-4-one, prepared according to Preparation 1 (23.1 g, 79 mmol) and 2,6-diisopropylphenol (42.2 g, 237 mmol) were dissolved in anhydrous N,N-dimethyl formamide (160 ml). Potassium carbonate (32.3 g, 234 mmol) was added and the mixture heated to 90° C. under nitrogen for 4.5 h. The mixture was then cooled, added to water (800 ml) and extracted with ethyl acetate (3×300 ml). The combined organic extracts were washed with sodium bicarbonate (300 ml), water (2×500 ml) and brine (300 ml), dried over magnesium sulphate and concentrated in vacuo. The residue was purified by column chromatography (Silica gel; dichloromethane then 2% methanol in dichloromethane. The isolated product was re-crystallised from ethyl acetate (250 ml) as a pale yellow solid. Residual ethyl acetate was removed by dissolving the compound in dichloromethane and concentrating in vacuo to give the title compound as a pale yellow solid. 20.5 g, 52%

| | |
|---|---|
| mpt.: | 123-126° C. |

(300 MHz, CDCl₃) δ 1.15(12H, d, 4×CH-Me); 3.0 (4H, m); 4.00 (6H, s, 2×OCH₃); 4.25 (2H, t, C₍₆₎H₂); 6.40 (1H, s, C₍₁₎H); 6.65 (1H, s, Ar—H); 7.20 (4H, m).

### EXAMPLE 3

### Synthesis of 2-(2,6-dimethylphenoxy)-6,7-dihydro-9,10-dimethoxy-4H-pyrimido-[6,1-a]isoquinolin-4-one

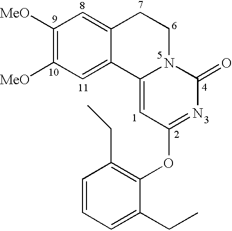

2-Chloro-6,7-dihydro-9,10-dimethoxy-4H-pyrimido-[6,1-a]isoquinolin-4-one, prepared according to Preparation 1 (1.0 g, 3.411 mmol), and 2,6-diethylphenol (1.53 g, 10.2 mmol) were dissolved in propan-2-ol (125 ml). Potassium carbonate (1.41 g, 10.2 mmol) was added and the mixture heated to reflux under nitrogen for 24 h. The mixture was then cooled and the solid removed by filtration. The filtrate was evaporated in vacuo and the residue purified by column chromatography (Silica gel; 2:1 ethyl acetate : dichloromethane) to give the title compound as a pale yellow solid. 1.21 g, 88%

| | | |
|---|---|---|
| mpt.: | 180-181° C. | |
| HPLC: | Area (%) | 100.00 |
| | Column | ODS (150 × 4.6 mm) |
| | MP | pH = 4 KH₂PO₄/MeOH (50/50) |
| | FR | 0.8 ml/min |
| | R_{T} | 11.634 |
| | Detection | 250 nm |

(300 MHz, CDCl₃) δ 1.21 (6H, t, CH₃); 2.54 (4H, q, CH₂); 2.99 (2H, t, J=6.5 Hz, C₍₇₎H₂); 3.98 (3H, s, OCH₃); 4.00 (3H, s, OCH₃); 4.22 (2H, t, J=6.5 Hz, C₍₆₎H₂); 6.42 (1H, s, C₍₁₎H); 6.79 (1H, s, Ar—H); 7.10-7.19 (3H, m, Ar—H); 7.22 (1H, s, Ar—H).

### EXAMPLE 4

### Synthesis of 6,7-Dihydro-9,10-dimethoxy-2-(2,4,6-trimethylphenoxy)-4H-pyrimido-[6,1-a]isoquinolin-4-one

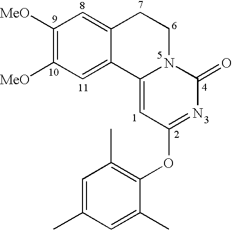

2-Chloro-6,7-dihydro-9,10-dimethoxy-4H-pyrimido-[6,1-a]isoquinolin-4-one, as prepared in Preparation 1 (1.2 g, 4.10 mmol), and 2,4,6-trimethylphenol (obtained from Aldrich) (1.68 g, 12.34 mmol) were dissolved in propan-2-ol (145 m). Potassium carbonate (1.70 g, 12.30 mmol) was added and the mixture heated under nitrogen for 16 h. After cooling to room temperature, the mixture was filtered and the precipitate washed with acetone. The combined filtrates were evaporated in vacuo. The residue was dissolved in dichloromethane (200 ml) and washed with 10% sodium hydroxide _{(aq)} (40 ml) and then water (40 ml). The organic layer was dried over magnesium sulphate, filtered and evaporated in vacuo to yield a brown oil. Column chromatography (Silica gel: 4:1 ethyl acetate:petroleum ether 40-60° followed by 4:1 ethyl acetate:dichloromethane) gave a yellow solid. This material was washed with diethyl ether, collected by filtration and dried in vacuo to yield the title compound as a pale yellow solid. 0.56 g, 35%.

| | | | |
|---|---|---|---|
| mpt.: | 230-231° C. | | |
| m/z: | C₂₃H₂₄N₂O₄ | | found |
| | requires M = 392 | | (M + 1) = 393 |
| HPLC: | Area (%) | 99.65 | |
| | Column | ODS (150 × 4.6 mm) | |
| | MP | 0.2 M NH₄Ac/ | |
| | | MeOH (30/70) | |
| | FR (ml/min) | 0.7 | |
| | R_{T} (min) | 13.006 | |
| | Detection | 250 nm | |

(300 MHz, CDCl₃) δ 2.15 (6H, s, 2×CH₃); 2.28 (3H, s, CH₃); 3.0 (2H, t, C₍₇₎H₂); 3.98 (6H, s, 2×OCH₃); 4.23 (2H, t, C₍₆₎H₂); 6.42 (1H, s, C₍₁₎H); 6.78 (1H, s, Ar—H); 6.86 (2H, s, 2×Ar—H); 7.20 (1H, s, Ar—H).

### EXAMPLE 5

### Synthesis of 2-(4-Chloro-2,6-dimethylphenoxy)-6,7-dihydro-9,10-dimethoxy-4H-pyrimido-[6,1-a]isoquinolin-4-one

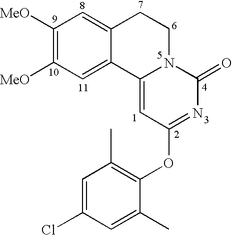

2-Chloro-6,7-dihydro-9,10-dimethoxy-4H-pyrimido-[6,1-a]isoquinolin-4-one, prepared in Preparation 1, (1.5 g, 5.13 mmol) and 4-chloro-2,6-dimethylphenol (obtained from Lancaster Chemicals) (2.41 g, 15.39 mmol) were dissolved in propan-2-ol (180 ml). Potassium carbonate (2.13 g, 15.41 mmol) was added and the mixture heated under nitrogen for 16 h. After cooling to room temperature, the mixture was filtered and the precipitate washed with acetone. The combined filtrates were evaporated in vacuo. The residue was dissolved in dichloromethane (200 ml) and washed with 10% sodium hydroxide _{(aq)} (45 ml) and then water (45 ml). The organic layer was dried over magnesium sulphate, filtered and evaporated in vacuo to yield an orange oil. Column chromatography (Silica gel: 4:1 ethyl acetate:dichloromethane) gave a yellow solid. This material was washed with diethyl ether, collected by filtration and dried in vacuo to yield the title compound as a pale yellow powder. 1.78 g, 84%.

| | | | |
|---|---|---|---|
| mpt.: | 201-203° C. | | |
| m/z: | C₂₂H₂₁³⁵ClN₂O₄ | | found |
| | requires M = 412 | | (M + 1) = 413 |
| | C₂₂H₂₁³⁷ClN₂O₄ | | found |
| | requires M = 414 | | (M + 1) = 415 |
| HPLC: | Area (%) | 97.91 | |
| | Column | ODS | |
| | | (250 × 4.6 mm) | |
| | MP | 0.1 M NH₄ | |
| | | Ac/MeCN (40/60) | |
| | FR (ml/min) | 0.7 | |
| | R_{T} (min) | 12.466 | |
| | Detection | 250 nm | |

(300 MHz, CDCl₃) δ 2.13 (6H, s, 2×CH₃); 2.99 (2H, t, C₍₇₎H₂); 3.98 (3H, s, OCH₃); 4.02 (3H, s, OCH₃); 4.23 (2H, t, C₍₆₎H₂); 6.43 (1H, s, C₍₁₎H); 6.78 (1H, s, Ar—H); 7.04 (2H, s, 2×Ar—H); 7.21 (1H, s, Ar—H).

### EXAMPLE 6

### Synthesis of 2-(4-Bromo-2,6-dimethylphenoxy)-6,7-dihydro-9,10-dimethoxy-4H-pyrimido-[6,1-a]isoquinolin-4-one

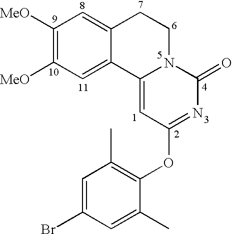

2-Chloro-6,7-dihydro-9,10-dimethoxy-4H-pyrimido-[6,1-a]isoquinolin-4-one, prepared in Preparation 1, (1.5 g, 5.13 mmol) and 4-bromo-2,6-dimethylphenol (obtained from Lancaster Chemicals) (3.1 g, 15.43 mmol) were dissolved in propan-2-ol (180 ml). Potassium carbonate (2.13 g, 15.41 mmol) was added and the mixture heated under nitrogen for 16 h. After cooling, to room temperature, the mixture was filtered and the precipitate washed with acetone. The combined filtrates were evaporated in vacuo. The residue was dissolved in dichloromethane (200 ml) and washed with 10% sodium hydroxide _{(aq)} (45 ml) and then water (45 ml). The organic layer was dried over magnesium sulphate, filtered and evaporated in vacuo to yield an orange oil. Column chromatography (Silica gel: 4:1 ethyl acetate:dichloromethane) gave a yellow solid. This material was washed with diethyl ether, collected by filtration and dried in vacuo to yield the title compound as a pale yellow powder 1.90 g, 81%.

| | | | |
|---|---|---|---|
| mpt.: | 232-234° C. | | |
| m/z: | C₂₂H₂₁⁷⁹BrN₂O₄ | | found |
| | requires M = 456 | | (M + 1) = 457 |
| | C₂₂H₂₁⁸¹BrN₉O₄ | | found |
| | requires M = 458 | | (M + 1) = 459 |
| HPLC: | Area (%) | 99.77 | |
| | Column | ODS (150 × 4.6 mm) | |
| | MP | 0.1 M NH₄ | |
| | | Ac/MeCN (57/43) | |
| | FR (ml/min) | 1.0 | |
| | R_{T} (min) | 11.565 | |
| | Detection | 250 nm | |

(300 MHz, CDCl₃) δ 2.14 (6H, s, 2×CH₃); 2.98 (2H, t, C₍₇₎H₂); 3.97 (3H, s, OCH₃); 3.99 (3H, s, OCH₃); 4.22 (2H, t, C₍₆₎H₂); 6.43 (1H, s, C₍₁₎H); 6.78 (1H, s, Ar—H); 7.19 (2H, s, 2×Ar—H); 7.21 (1H, s, Ar—H).

### Example 7

### Synthesis of 6,7-Dihydro-9,10-dimethoxy-2-(4-ethanoyl-2,6-dimethylphenoxy)-4H-pyrimido-[6,1-a]isoquinolin-4-one

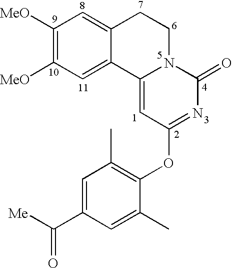

6,7-Dihydro-2-chloro-9,10-dimethoxy-4H-pyrimido-[6,1-a]isoquinolin-4-one, prepared in Preparation 1, (1.5 g, 5.13 mmol) and 2,6-dimethyl-4-hydroxyacetophenone (obtained from Maybridge Chemicals) (2.53 g, 15.41 mmol) were dissolved in propan-2-ol (180 ml). Potassium carbonate (2.13 g, 15.41 mmol) was added and the mixture heated under nitrogen for 23 h. After cooling to room temperature, the mixture was filtered and the precipitate washed with acetone. The combined filtrates were evaporated in vacuo. The residue was dissolved in dichloromethane (200 ml) and washed with water (2×45 ml). The organic layer was dried over magnesium sulphate, filtered and evaporated in vacuo to yield an orange oil. Column chromatography (Silica gel: 4:1 ethyl acetate:dichloromethane) gave a yellow solid. This material was washed with diethyl ether, collected by filtration and dried in vacuo to yield the title compound as a pale yellow powder 1.81 g, 84%.

| | | | | |
|---|---|---|---|---|
| mpt.: | 228-230° C. | | | |
| m/z: | C₂₄H₂₄N₂O₅ | | requires | found (M + |
| | | | M = 420 | 1) = 421 |
| HPLC: | Area (%) | 99.32 | | |
| | Column | ODS | | |
| | MP | 0.1M NH₄Ac/MeCN | | |
| | | (65/35) | | |
| | FR (ml/min) | 1.0 | | |
| | R_{T} (min) | 13.840 | | |
| | Detection | 250 nm | | |

(300 MHz, CDCl₃) δ 2.25 (6H, s, 2×CH₃); 2.60 (3H, s, CH₃CO); 3.02 (2H, t, C₍₇₎H₂); 3.99 (3H, s, OCH₃); 4.01 (3H, s, OCH₃); 4.23 (2H, t, C₍₆₎H₂); 6.47 (1H, s, C₍₁₎H); 6.78 (1H, s, Ar—H); 7.21 (1H, s, Ar—H); 7.68 (2H, s, 2×Ar—H).

### EXAMPLE 8

### Synthesis of 2-(2,6-Dichlorophenoxy)-6,7-dihydro-9,10-dimethoxy-4H-pyrimido-[6,1-a]isoquinolin-4-one

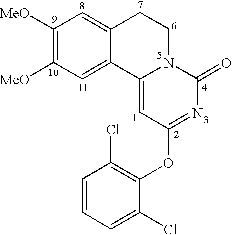

2-Chloro-6,7-dihydro-9,10-dimethoxy-4H-pyrimido-[6,1-a]isoquinolin-4one, prepared in Preparation 1 (25 g, 85 mmol) and 2,6-dichlorophenol (25.1 g, 153 mmol) were dissolved in anhydrous N,N-dimethyl formamide (150 ml). Potassium carbonate (30 g, 217 mmol) was added and the mixture heated to 90° C. under nitrogen for 2 h. The mixture was then cooled, partitioned between water (1 l) and ethyl acetate (500 ml). The resulting precipitate was filtered, washed with ethyl acetate (100 ml) and water (100 ml), re-dissolved in dichloromethane (500 ml) and washed with brine (200 ml), dried over magnesium sulphate and concentrated in vacuo. The residue was re-crystallised from acetonitrile (600 ml) to give the title compound as a pale yellow solid. 22 g, 53%

| | |
|---|---|
| mpt.: | 240-242° C. |

(300 MHz, d₆DMSO) δ 2.05 (2H, t, C₍₇₎H₂); 3.85 (3H, s, OCH₃); 3.90 (3H, s, OCH₃); 4.05 (2H, t, C₍₆₎H₂); 7.00 (1H, s, C₍₁₎H); 7.15 (1H, s, Ar—H); 7.35 (1H, t, Ar—H); 7.65 (1H, s, Ar—H).

### EXAMPLE 9

### Synthesis of 9,10-Dimethoxy-2-(2-methylphenoxy)-6,7-dihydro-4H-pyrido[6,1-a]isoquinolin-4-one

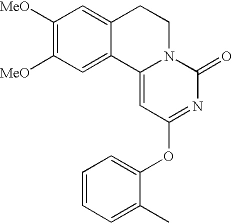

2-Chloro-9,10-dimethoxy-6,7-dihydro-4H-pyrimido[6,1-a]isoquinolin-4-one (1) (1.5 g, 5.13 mmol) and o-cresol (1.66 g, 15.4 mmol) were dissolved in iso-propanol (180 ml). Potassium carbonate (2.13 g, 15.4 mmol) was added and the mixture was heated at reflux, under nitrogen, for 18 h. After cooling to room temperature, the mixture was filtered and the precipitate washed with acetone. The combined filtrates were evaporated in vacuo and the residue was dissolved in dichloromethane (220 ml), washed with 10% NaOH (40 ml), then with water (40 ml) and dried (MgSO₄). Evaporation in vacuo gave a brown oil which was purified by column chromatography on silica gel (EtOAc/petroleum ether 80:20). The title compound was obtained as a pale yellow solid, 0.56 g, 35%.

| | | | |
|---|---|---|---|
| M.p.: | 201-203° C. | | |
| m/z: | C₂₁H₂₀N₂O₄ | | found |
| | requires M = 364 | | (M + l) = 365 |
| HPLC: | Area (%) | 99.01 | |
| | Column | ODS (150 × 4.6 mm) | |
| | MP | 0.2 M NH₄OAc/ | |
| | | MeOH (40/60) | |
| | FR (ml/min) | 0.8 | |
| | RT (min) | 14.976 | |
| | Detection | 235 nm | |

¹H NMR (300 MHz, CDCl₃): δ 2.22 (3H, s, ArCH₃), 2.97 (2H, t, CH₂), 3.98 (6H, s, 2×OCH₃), 4.21 (2H, t, CH₂), 6.40 (1H, s, C—CH), 6.78 (1H, s, ArH), 7.047.25 (5H, m, 5×ArH).

### EXAMPLE 10

### Synthesis of 9,10-Dimethoxy-2-(2-isobutylphenoxy-4-6,7-dihydro-4H-pyrimido[6,1-a]isoquinolin-4-one

2-Isobutylphenol

Potassium tert-butoxide (44.8 g, 400 mmol) was suspended in degassed heptane (1300 ml) and butyllithium (258 ml, 400 mmol) added. The reaction mixture was allowed to stir at room temperature for 20 min and then O-cresol (10.8 g, 100 mmol) added followed by heating at reflux for 3 hours. The mixture was then cooled to room temperature and the precipitate allowed to settle. The excess solvent was decanted off and the residue washed with pentane. This process was repeated once more and the resultant precipitate suspended in THF (1500 ml). This suspension was transferred, via cannula, to a solution of isopropylbromide (10.33 ml, 110 mmol) in TV (500 ml) and the reaction mixture stirred at room temperature overnight. The mixture was quenched by addition of water (50 ml) and acidified with HCl (5M). The aqueous phase was separated and extracted with chloroform (3×100 ml). The organic phases were combined, dried (MgSO₄), filtered and concentrated to give a red brown liquid. The crude material was purified by flash column chromatography (90:10 petrol:EtOAc) to give the title compound as a yellow oil (7.63 g, 51%).

9,10-Dimethoxy-2-(2-isobutylphenoxy)-6,7-dihydro-4H-pyrimido[6,1-a]isoquinolin-4-one

2-Isobutylphenol (1.24 g, 8.16 mmol) was dissolved in THF (40 ml) and cooled to −78° C. Butyllithum (5.12 ml, 8.16 mmol) was then added and the reaction mixture stirred at −78° C. for 1 hour and then slowly warmed to room temperature overnight. The reaction was quenched by addition of NH₄Cl (40 ml) and the mixture extracted with EtOAc (3×50 ml). The combined organic phases were washed with NH₄Cl (50 ml). This aqueous phase was combined with that from the previous washing and extracted with EtOAc (50 ml). All the organic phases were now combined and washed with brine, dried (MgSO₄), filtered and concentrated to give a yellow oily solid Ether was added to the residue and the resultant solid filtered off and washed with ice cold ether. The residue was purified by flash column chromatography eluting with ethyl acetate to give the title compound as a white solid (0.5;, 18%).

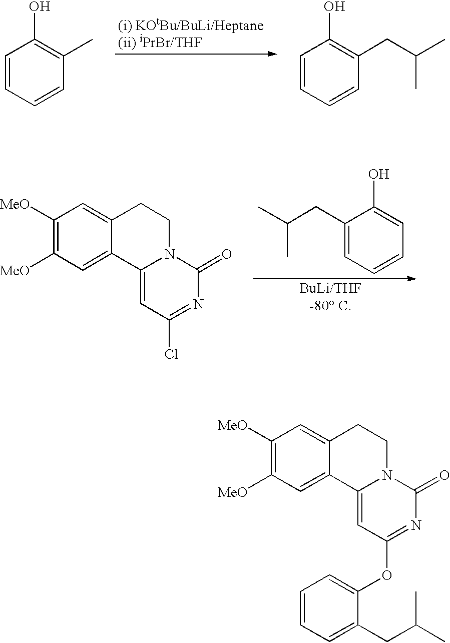

### EXAMPLE 11

### Synthesis of 2-(2-tert-butylphenoxy)-9,10-dimethoxy-6,7-dihydro-4H-pyrimido[6,1-a]isoquinolin-4-one

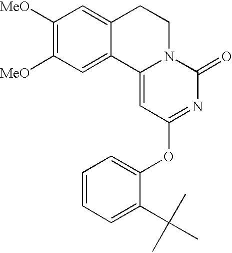

2-Chloro-9,10-dimethoxy-6,7-dihydro-4H-pyrimido[6,1-a]isoquinolin-4-one (1.47 g, 5.0 mmol) and 2-tert-butylphenol (2.25 g, 15.0 mmol) were dissolved in 2-propanol (180 ml). Potassium carbonate (2.07 g, 15.0 mmol) was added and the mixture was stirred and heated to reflux, under nitrogen, for 6 h. After cooling to room temperature the mixture was filtered and the filtrate was evaporated in vacuo. The residue was purified by column chromatography on silica gel [ethyl acetate/dichloromethane (3:2)] to provide the above compound (1.35 g, 66%) as an off-white solid.

| | | | |
|---|---|---|---|
| M.p.: | 226-228° C. | | |
| M/z: | C₂₄H₂₆N₂O₄ | | found m/z |
| | requires M = 406, | | [ES+] = 407 |
| HPLC: | Area (%) | 99.68 | |
| | Column | S5 C8 (250 × 4.6 mm) | |
| | MP | pH 4 0.02 M KH₂PO₄/ | |
| | | CH₃CN (35/65) | |
| | RT (min) | 9.175 | |
| | FR (ml/min) | 0.7 | |
| | Detection | 254 nm | |

¹H NMR (300 MHz, CDCl₃): δ 1.39 (9H, s, CH(CH)₃), 2.97 (2H, t, CH₂), 3.97 (3H, s, OCH₃), 3.98 (3H, s, OCH₃), 4.23 (2H, t, CH₂), 6.38 (1H, s, C═CH), 6.78 (1H, S, ArH), 7.05-7.27 (4H, m, 4×ArH), 7.40 (1H, m, ArH).

### EXAMPLE 12

### Synthesis of 9,10-Dimethoxy-2-(2-ethylphenoxy)-6,7-dihydro-4H-pyrimido[6,1-a]isoquinolin-4-one

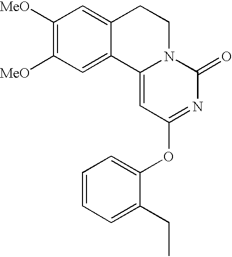

2-Chloro-9,10-dimethoxy-6,7-dihydro-4H-pyrimido[6,1-a]isoquinolin-4-one (1.47 g, 5.0 mmol) and 2-ethylphenol (1.22 g, 10.0 mmol) were dissolved in 2-propanol (180 ml). Potassium carbonate (1.38 g, 10.0 mmol) was added and the mixture was stirred and heated to reflux, under nitrogen, for 6 h. After cooling to room temperature the mixture was filtered and the filtrate was evaporated in vacuo. The residue was purified by column chromatography on silica gel [ethyl acetate/dichloromethane (3:2)] to provide the above compound (1.27 g, 67%) as a pale yellow solid.

| | | | |
|---|---|---|---|
| M.p.: | 167-169° C. | | |
| M/z: | C₂₂H₂₂N₂O₄ | | found m/z |
| | requires M = 378, | | [ES+] = 379 |
| HPLC: | Area (%) | 99.46 | |
| | Column | S5 C8 (250 × 4.6 mm) | |
| | MP | pH 4 0.02 M KH₂PO₄/ | |
| | | CH₃CN (35/65) | |
| | RT (min) | 7.725 | |
| | FR (ml/min) | 0.7 | |
| | Detection | 254 mn | |

¹H NMR (300 MHz, CDCl₃): δ 1.21 (3H, t, CH₂CH₃), 2.59 (2H, q, CH₂CH₃), 2.97 (2H, t, CH₂), 3.97 (6H, s, 2×OCH₃), 4.21 (2H, t, CH₂), 6.41 (1H, s, C═CH), 6.78 (1H, s, ArH), 7.05-7.28 (5H, m, 5×ArH).

### EXAMPLE 13

### Synthesis of 2-(2-Cyclopentylphenoxy)-9,10-dimethoxy-6.7-dihydro-4H-pyrimido[6,1-a]-isoquinolin-4-one

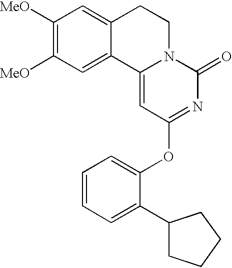

2-Chloro-9,10-dimethoxy-6,7-dihydro-4H-pyrimido[6,1-a]isoquinolin-4-one (1.20 g, 4.11 mmol) and 2-cyclopentylphenol (1.0 g, 6.17 mmol) were dissolved in 2-propanol (150 ml). Potassium carbonate (1.14 g, 8.23 mmol) was added and the mixture was stirred and heated to reflux, under nitrogen, for 18 h. After cooling to room temperature the mixture was filtered and the filtrate was evaporated in vacuo. The residue was purified by column chromatography on silica gel [ethyl acetate:dichloromethane (3:2)] to provide the above compound (0.51 g, 30%) as a pale yellow solid.

| | | | |
|---|---|---|---|
| M.p.: | 122-125° C. | | |
| M/z: | C₂₅H₂₆N₂O₄ | | found m/z |
| | requires M = 418, | | [ES +] = 419 |
| HPLC: | Area (%) | 99.43 | |
| | Column | S5 C8 (250 × 4.6 mm) | |
| | MP | pH 4 0.02 M KH₂PO₄/ | |
| | | CH₃CN (35/65) | |
| | RT (min) | 10.191 | |
| | FR (ml/min) | 0.7 | |
| | Detection | 254 nm | |

¹H NMR (300 MHz, CDCl₃): δ 1.57-2.05 (5H, m, cyclopentyl 4×CH₂), 2.97 (2H, t, CH₂), 3.13 (1H, m, ArCH), 3.97 (6H, s, 2×OCH₃), 4.21 (2H, t, CH₂), 6.38 (1H, s, C═CH), 6.78 (1H, s, ArH), 7.07 (1H, m, ArH), 7.18 (3H, m, 3×ArH), 7.32 (1H, m, ArH).

The pharmacological utility of the compounds of the present invention has been demonstrated in studies using compounds previously synthesised as described in the above Examples. The results below serve to illustrate the generic application of the compounds of the present invention.

### EXAMPLE A

### Efficacy of Compounds of the Invention Against Electrical-Induced Contraction of Guinea-Pig Isolated Trachea

The efficacy of compounds of the invention was tested against electrical-induced contraction of guinea-pig isolated trachea. The results demonstrate that the compounds of Examples 2, 3, 10, 11, 12 and 13 inhibit the contractile responses with a long duration of action.

### Example A (i)

### Superfusion Experiments

Method

Superfusion of guinea-pig tracheal rings was performed according to a previously described method (Coleman et al. Pulmonary Pharmacology 9 107-117 (1996)). Briefly, guinea-pig tracheal preparations were cut into rings and then opened by sectioning the ring opposite the smooth muscle and suspended between two platinum electrodes under 1 g tension. The tissues were superfused at a rate of from 1 to 3.25 ml/min with Krebs-Henseleit solution at 37° C. containing the cyclooxygenase inhibitor, indomethacin (5 μM) and bubbled with 95% O₂ and 5% CO₂. Tracheal preparations were allowed to equilibrate for 40 minutes before commencement of electrical stimulation, delivered as a 10 s train of square wave pulses at 3 Hz, 0.1 ms duration and 20V (approx 400 mamps) generated every 100 sec by means of physiological square wave-stimulator. The compounds of the present invention were superfused at a rate of 0.2 to 0.3 ml/min ad contractile responses to electrical field stimulation were recorded on a Macintosh computer using MacLab sor vare. The drug was prepared in DMSO and diluted in Krebs-Henseleit solution which yielded a final superfusion concentration of 0.05 to 0.1% DMSO.

Results

The duration of action was determined and expressed as onset time (OT₅₀) and recovery time (RT₅₀). The time taken to reach 50% of the maximum inhibition of the contractile response (onset time: OT) was determined and similarly, the time taken for a 50% reversal of the maximum inhibition of the contractile response (recovery time: RT₅₀) following the termination of exposing the tissue to compound. These values are shown in Table 1.

As shown in Table 1, all the tested compounds caused a time dependent inhibition of the contractile response to electrical field stimulation in guinea-pig isolated trachea. The recovery of the contractile response to electrical field stimulation was slow, with the inhibitory responses not reversing 4 hours after cessation of administration of the drug. The vehicle, DMSO, failed significantly to inhibit the contractile response to electrical field stimulation (FIG. 1).

TABLE 1
OT₅₀ and RT₅₀ values

| | | RT₅₀ | |
|---|---|---|---|
| Compound | OT₅₀ (min) | (min) | Figure |
| 2-(2,6-diethylphenoxy)-6,7-dihydro-9,10- | 17 ± 1 | >240 | |
| dimethoxy-4H-pyrimido-[6,1-a] | | | |
| isoquinolin-4-one (Example 3) | | | |
| 2-(2-tert-butylphenoxy)-9,10-dimethoxy-6, | 14.7 ± 1.33 | >300 | |
| 7-dihydro-4H-pyrimido[6,1-a]isoquinolin- | | | |
| 4-one (Example 11) | | | |
| 9,10-Dimethoxy-2-(2-ethylphenoxy)-6,7- | 9.7 ± 0.6 | 263 | |
| dihydro-4H-pyrimido[6,1-a]isoquinolin- | | | |
| 4-one (Example 12) | | | |
| 2-(2-Cyclopentylphenoxy)-9,10- | 18.6 ± 1.5 | >300 | |
| dimethoxy-6,7-dihydro-4H-pyrimido[6, | | | |
| 1-a]-isoquinolin-4-one (Example 13) | | | |
| 9,10-Dimethoxy-2-(2-isobutylphenoxy)-6, | 17.5 ± 1.25 | >300 | |
| 7-dihydro-4H-pyrimido[6,1-a]isoquinolin- | | | |
| 4-one (Example 10) | | | |
| 6,7-Dihydro-2-(2,6-diisopropylphenoxy)- | 24.9 ± 5.0 | >240 | |
| 9,10-dimethoxy-4H-pyrimido-[6,1-a] | | | |
| isoquinolin-4-one (Example 2) | | | |
| Trequinsin (9,10-dimethoxy-3-methyl-2- | 14 | 146 | |
| mesitylimino-2,3,6,7-tetrahydro-4H- | | | |
| pyrimido[6,1-a]isoquinolin-4-one) | | | |

(Each value is the mean and s.e. mean of three observations except where otherwise indicated, as “n”)

### Example A (ii)

### Organ Bath Experiments

Method

Guinea-pig tracheal rings were suspended in organ baths under 1 g tension between two electrodes and subjected to electrical field stimulation (3 Hz, 0.1 ms duration and 20V (approx 400 mAmps) generated every 100 sec by means of physiological square wave-stimulator. The compounds of Examples 1, 2 and 8 were dissolved in DMSO containing Tween 80 (10%) and distilled water (0.01M), which were then added to the organ bath to give a final concentration of 10 μM.

Results

The results are shown in FIGS. 6 (for compound of Example 1), 7 (for compound of Example 8) and 8 (for compound of Example 2). All compounds caused complete inhibition of the contractile response to electrical field stimulation and the effect was maintained for more than 2-4 hours.

### EXAMPLE B

### Efficacy of Compounds of the Invention Against Proliferation of Human Mononuclear Cells Stimulated by PHA

The effect of compounds of the invention against proliferation of human mononuclear cells stimulated by PHA was also investigated. Proliferation was significantly inhibited by these compounds, indicating that they possess anti-inflammatory activity. The results below serve to illustrate the generic application of the novel compounds of the present invention.

Method

Normal healthy volunteers underwent phlebotomy and 25 ml of blood was collected. Mononuclear cells were separated and purified according to the method of Banner et al. (Banner et al. Br. J. Pharmacol. 116 3169-3174 (1995)). Human peripheral mononuclear cells (100,000 per well) were stimulated for 24 h with phytohaemagglutinin (PHA, 2 μg/ml) in the absence or presence of the compounds of Examples 1, 2 and 8 (0.001-100 μM) at 37° C. in a 95% air, 5% CO₂ atmosphere. Twenty four hours later, [³H]-thymidine (0.1 μCi) was added to each well and the cells were incubated for a further 24 h period. Cells were then harvested onto glass fibre filters using a cell harvester (ICN Flow, Buckinghamshire) and counted in a scintillation counter.

Results

All the compounds under test caused a concentration dependent inhibition of the proliferation of human mononuclear cells stimulated with PHA (n=6; FIG. 9). The IC₅₀ values and 95% confidence limits for these compounds are indicated in Table 2. The results are also shown in the graph of FIG. 10.

Table 2: IC₅₀ Values for Various Compounds Against Proliferation of Human Mononuclear Cells Stimulated with PHA (n=6)

| Compound | IC₅₀ |
|---|---|
| 6,7-Dihydro-2-(2,6-dimethylphenoxy)-9,10- | 5.13 μM |
| dimethoxy-4H-pyrimido-[6,1-a]isoquinolin-4-one | (2.88-9.14) |
| (Examp1e 1) | |
| 6,7-Dihydro-2-(2,6-diisopropylphenoxy)-9,10- | 2.90 μM |
| dimethoxy-4H-pyrimido-[6,1-a]isoquino1in-4-one | (1.56-5.41) |
| (Example 2) | |
| 2-(2,6-Dichlorophenoxy)-6,7-dihydro-9,10- | 8.22 μM |
| dimethoxy-4H-pyrimido-[6,1-a]isoquinolin-4-one | (4.23-16.0) |
| (Example 8) | |

### EXAMPLE C

6,7-Dihydro-2-(2,6-diisopropylphenoxy)-9,10-dimethoxy-4H-pyrimido-[6,1-a]isoquinolin-4-one (compound of Example 2) has been shown to be a potent inhibitor of phosphodiesterase (PDE) type 3 and 4 isozymes. The IC₅₀ value is shown below.

| | PDE3 (nM) | PDE4 (nM) |
|---|---|---|
| | (human platelet) | (human neutrophil) |
| Example 2 | 107 | 1195 |
| Rolipram | ND | 6412 |
| Cilostamide | 89 | ND |

Rolipram is a known PDE 4 inhibitor and cilostamide is a known PDE3 inhibitor
ND—Not determined

### EXAMPLE D

6,7-Dihydro-2-(2,6-diisopropylphenoxy)-9,10-dimethoxy-4H-pyrimido-[6,1-a]isoquinolin-4-one (compound of Example 2) was tested for effects on LPS induced TNF-α release from human monocytes. Results are below.

| | IC₅₀ (nM) | |
|---|---|---|
| Compound | 250 | n = 6 |
| CDP 840 (PDE4 inhibitor) | 92 | n = 6 |
| Siguazodan (PDE3 inhibitor) | >100 | μM |

### EXAMPLE E

### In vivo Tests on 6,7-Dihydro-2-(2,6-diisopropylphenoxy)-9,10-dimethoxy-4H-pyrimido-[6,1-a]isoquinolin-4-one (Compound of Example 2)

1. The above compound was tested in a model of histamine induced bronchospasm. Conscious guinea-pigs were exposed to dry powder (micronised) of the compound. The drug was blended with lactose so that the final concentration was 0.25, 2.5 and 25%. At various times after exposure to drug the animals were anaesthetised and challenged with varying doses of histamine. Total airway resistance and mean arterial blood pressure were recorded. Exposure to dry powder (25%) provided significant protection against histamine induced bronchospasm without changing mean arterial blood pressure.

2. Intravenous administration of the compound of Example 2 (0.1 to 300 μg/kg) to urethane anaesthetised guinea-pigs produced a dose dependant reduction in mean arterial blood pressure. The compound was dissolved in DMSO then diluted with saline (there was evidence that the compound had come out of solution).

3. In a model of antigen induced eosinophilia in the ovalbumin sensitised guinea-pig, the compound of Example 2 (10 mg/kg) administered orally 1 hour prior to antigen challenge, significantly inhibited the recruitment of eosinophils to the lungs following antigen challenge (aerosol) in sensitised guinea-pigs.

### EXAMPLE F

### Taste of Compounds

For pharmaceutical compounds which are administered orally, how the compounds taste is a very important factor in ensuring patient compliance. Unexpectedly, the compounds of the present invention are substantially tasteless. They are therefore particularly suitable for oral administration, for example as dry powder to be inhaled.

Method

Small amounts of all the compounds of the above Examples 1 to 13, trequinsin (9,10-dimethoxy-3-methyl-2-mesitylimino-2,3,6,7-tetrahydro-4H-pyrimido[6,1-a]isoquinolin-4-one) and desmethyl trequinsin (9,10-dimethoxy-2-mesitylimino-2,3,6,7-tetrahydro-4H-pyrimido[6,1-a]isoquinolin-4-one) were placed on the tip of the tongue of an informed, healthy male volunteer and the taste of each compound was assessed.

Results

All the compounds of Examples 1 to 13 were not bitter, and therefore have significantly improved taste compared with trequinsin or desmethyl trequinsin, which are both very bitter.

1. A compound of general formula I: 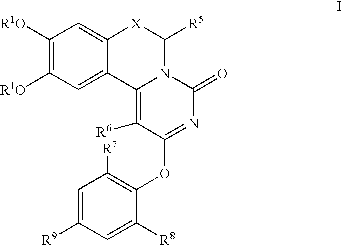
wherein
each of R¹and R² independently represents a C₁₋₆ alkyl or C₂₋₇ acyl group;
X represents OCH₂ or a group CR³R⁴, wherein each of R³ and R⁴ independently represents a hydrogen atom or a C₁₋₃ alkyl group;
R⁵ represents a hydrogen atom or a C₁₋₃ alkyl, C₂₋₃ alkenyl or C₂₋₃ alkynyl group;
R⁶ represents a hydrogen atom or a C₁₋₆ alkyl, C₂₋₆ alkenyl, C₂₋₆ alkynyl, amino, C₁₋₆ alkylamino, di(C₁₋₆) alkylamino or C₂₋₇ acylamino group;
each of R⁷ and R⁸ independently represents a hydrogen or halogen atom or a hydroxy, trifluoromethyl, C₁₋₆ alkyl, C₂₋₆ alkenyl, C₂₋₆ alkynyl, C₂₋₇ acyl, C₁₋₄ alkylthio, C₁₋₆ alkoxy or C₃₋₆ cycloalkyl group;
R⁹ represents a hydrogen or halogen atom or a hydroxy, trifluoromethyl, C₁₋₆ alkyl, C₂₋₆ alkenyl, C₂₋₆ alkynyl, C₂₋₇ acyl, C₁₋₆ alkylthio, C₁₋₆ alkoxy or C₃₋₆ cycloalkyl group;
or a salt thereof;
excluding the compound 6,7-dihydro-2-phenoxy-9,10-dimethoxy-4H-pyrimido[6,1a]isoquinolin-4-one.

2. A compound as claimed in claim 1 wherein, independently or in any compatible combination,
each of R¹and R² represents a C₁₋₆ alkyl, preferably a C₁₋₄ alkyl, group;
R¹and R² are the same as each other;
each of R³ and R⁴ represents a hydrogen atom;
R⁵ represents a hydrogen atom;
R⁶ represents a hydrogen atom;
each of R⁷ and R⁸ represents a C₁₋₆ alkyl, preferably methyl, ethyl or isopropyl, group;
R⁷ and R⁸ are the same as each other;
R⁹ represents a halogen atom or a methyl or acetyl group.

3. 6,7-Dihydro-2-(2,6-dimethylphenoxy)-9,10-dimethoxy-4H-pyrimido-[6,1-a]iso-quinolinone.

4. 2-(2,6-Dimethylphenoxy)-6,7-dihydro-9,10-dimethoxy-4H-pyrimido-[6,1-a]iso-quinolinone.

5. 6,7-Dihydro-2-(2,6-diisopropylphenoxy)-9,10-dimethoxy-4H-pyrimido-[6,1-a]isoquinolin-4one.

6. 6,7-Dihydro-9,10-dimethoxy-2-(2,4,6-trimethylphenoxy)-4H-pyrimido-[6,1-a]isoquinolin one.

7. 2-4-Chloro-2,6-dimethylphenoxy)-6,7-dihydro-9,10-dimethoxy-4H-pyrimido-[6,1-a]isoquinolin-4-one.

8. 2-(4-Bromo-2,6-dimethylphenoxy)-6,7-dihydro-9,10-dimethoxy-4H-pyrimido-[6,1-a]isoquinolin-4-one.

9. 6,7-Dihydro-9,10-dimethoxy-2-(4-ethanoyl-2,6-dimethylphenoxy)-4H-pyrimido-[6,1-a]isoquinolin-4one.

10. 2-(2,6-Dichlorophenoxy)-6,7-dihydro-9,10-dimethoxy-4H-pyrimido-[6,1-a]isoquinolin-4-one.

11. 9,10-Dimethoxy-2-(2-methylphenoxy)-6,7-dihydro-4H-pyrimido[6,1-a]isoquinolin-4-one

12. 9,10-Dimethoxy-2-(2-isobutylphenoxy)-6,7-dihydro-4H-pyrimido[6,1-a]isoquinolin-4-one.

13. 2-(2-tert-butylphenoxy)-9,10-dimethoxy-6,7-dihydro-4H-pyrimido[6,1-a]isoquinolin-4-one.

14. 9,10-Dimethoxy-2-(2-ethylphenoxy)-6,7-dihydro-4H-pyrimido[6,1-a]isoquinolin-4-one.

15. 2-(2-Cyclopentylphenoxy)-9,10-dimethoxy-6,7-dihydro-4H-pyrimido[6,1-a]isoquinolin-4one.

16. A process for preparing a compound of general formula I as defined in claim 1, excluding the compound 6,7-dihydro-2-phenoxy-9,10-dimethoxy-4H-pyrimido[6,1-a]isoquinolin, the process comprising:
(a) reacting a compound of general formula II: 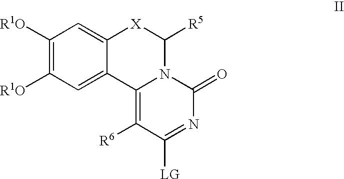
wherein R¹, R², R⁵, R⁶ and X are as defined for general formula I and LG represents a leaving group, with a compound of general formula III 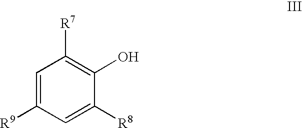
wherein R⁷, R³ and R⁹ are as defined for general formula I; or
(b) when X in general formula I represents a group CR³R⁴, wherein R³ represents a hydrogen atom, R⁴ represents a hydrogen atom or a C₁₋₃ alkyl group, and R⁵ represents a hydrogen atom or a C₁₋₃ alkyl group, hydrogenating, a compound of general formula IX: 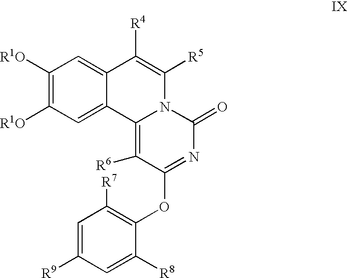
wherein R¹, R², R⁶, R⁷, R⁸ and R⁹ are as defined for general formula I; and
(c) optionally converting a compound of general formula I so formed into another compound of general formula I.

17. A process as claimed in claim 16, wherein LG in general formula II represents a chlorine atom.

18. A process as claimed in claim 16 or claim 17, wherein step (a) is carried out in a suitable solvent such as dimethylformamide or isopropanol in the presence of a base such as potassium carbonate.

19. A process as claimed in claims 16, 17 or 18 wherein the compound of general formula I is as defined in any of claim 1 to 15.

20. A composition comprising a compound of general formula I: 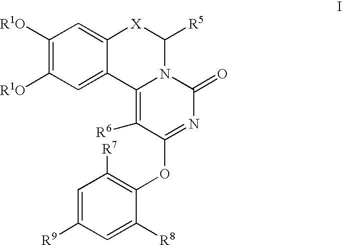
wherein
each of R¹and R² independently represents a C₁₋₆ alkyl or C₂₋₇ acyl group;
X represents OCH₂ or a group CR³R⁴, wherein each of R³ and R⁴ independently represents a hydrogen atom or a C₁₋₃ alkyl group;
R⁵ represents a hydrogen atom or a C₁₋₃ alkyl, C₂₋₃ alkenyl or C₂₋₃ alkynyl group;
R⁶ represents a hydrogen atom or a C₁₋₆ alkyl, C₂₋₆ alkenyl, C₂₋₆ alkynyl, amino, C₁₋₆ alkylamino, di(C₁₋₆) alkylamino or C₂₋₇ acylamino group;
each of R⁷ and R⁸ independently represents a hydrogen or halogen atom or a hydroxy, trifluoromethyl, C₁₋₆ alkyl, C₂₋₆ alkenyl, C₂₋₆ alkynyl, C₂₋₇ acyl, C₁₋₆ alkylthio, C₁₋₆ alkoxy or C₃₋₆ cycloalklyl group;
R⁹ represents a hydrogen or halogen atom or a hydroxy, trifluoromethyl, C₁₋₆ alkyl, C₂₋₆ alkenyl, C₂₋₆ alkynyl, C₂₋₇ acyl, C₁₋₆ alkylthio, C₁₋₆ alkoxy or C₃₋₆ cycloalkyl group;
or a salt thereof;
and a veterinarily or pharmaceutically acceptable carrier or diluent.

21. A composition as claimed in claim 20, further comprising an active agent such as a β₂-adrenoceptor agonist or a glucocorticoid steroid.

22. A composition as claimed in claim 20 or claim 21, wherein the composition is a pharmaceutical composition for human medicine.

23. A composition as claimed in claim 20, 21 or 22, adapted for administration by aerosol.

24. A composition as claimed in any of claims 20 to 23, wherein the compound is as defined in any of claims 1 to 15.

25. A compound of general formula I 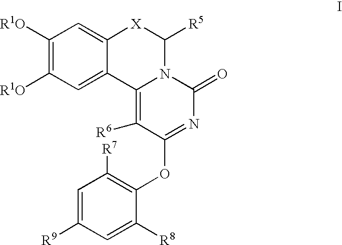
wherein
each of R¹and R² independently represents a C₁₋₆ alkyl or C₂₋₇ acyl group;
X represents OCH₂ or a group CR³R⁴, wherein each of R³ and R⁴ independently represents a hydrogen atom or a C₁₋₃ alkyl group;
R⁵ represents a hydrogen atom or a C₁₋₃ alkyl, C₂₋₃ alkenyl or C₂₋₃ alkynyl group;
R⁶ represents a hydrogen atom or a C₁₋₆ alkyl, C₂₋₆ alkenyl, C₂₋₄ alkynyl, amino, C₁₋₆ alkylamino, di(C₁₋₆) alkylamino or C₂₋₇ acylamino group;
each of R⁷ and R⁸ independently represents a hydrogen or halogen atom or a hydroxy, trifluoromethyl, C₁₋₆ alkyl, C₂₋₆ alkenyl, C₂₋₆ alkynyl, C₂₋₇ acyl, C₁₋₄ alkylthio, C₁₋₆ alkoxy or C₃₋₆ cycloalkyl group;
R⁹ represents a hydrogen or halogen atom or a hydroxy, trifluoromethyl, C₁₋₆ alkyl, C₂₋₆ alkenyl, C₂₋₆ alkynyl, C₂₋₇ acyl, C₁₋₆ alkylthio, C₁₋₆ alkoxy or C₃₋₆ cycloalkyl group;
or a salt thereof;
for use in medicine.

26. A compound of general formula I: 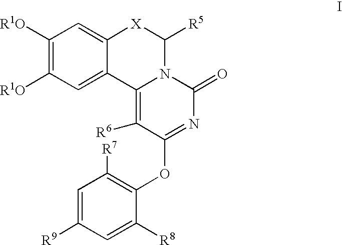
wherein
each of R¹ and R² independently represents a C₁₋₆ alkyl or C₂₋₇ acyl group;
X represents OCH₂ or a group CR³R⁴, wherein each of R³ and R⁴ independently represents a hydrogen atom or a C₁₋₃ alkyl group;
R⁵ represents a hydrogen atom or a C₁₋₃ alkyl, C₂₋₃ alkenyl or C₂₋₃ alkynyl group;
R⁶ represents a hydrogen atom or a C₁₋₆ alkyl, C₂₋₆ alkenyl, C₂₋₆ alkynyl amino, C₁₋₆ alkylamino, di(C₁₋₆ alkylamino or C₂₋₇ acylamino group;
each of R⁷ and R⁸ independently represents a hydrogen or halogen atom or a hydroxy, trifluoromethyl, C₁₋₆ alkyl, C₂₋₆ alkenyl, C₂₋₆ alkynyl, C₂₋₇ acyl, C₁₋₆ alkylthio, C₁₋₆ alkoxy or C₃₋₆ cycloalkyl group;
R⁹ represents a hydrogen or halogen atom or a hydroxy, trifluoromethyl, C₁₋₆ alkyl, C₂₋₆ alkenyl, C₂₋₆ alkyl, C₂₋₇ acyl, C₁₋₆ alkylthio, C₁₋₆ alkoxy or C₃₋₆ cycloalkyl group;
or a salt thereof;
for use as an inhibitor of a phosphodiesterase isoenzyme.

27. A compound of general formula I: 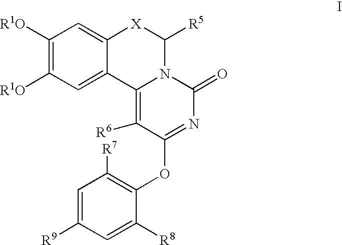
wherein
each of R¹and R² independently represents a C₁₋₆ alkyl or C₂₋₇ acyl group;
X represents OCH₂ or a group CR³R⁴, wherein each of R³ and R⁴ independently represents a hydrogen atom or a C₁₋₃ alkyl group;
R⁵ represents a hydrogen atom or a C₁₋₃ alkyl, C₂₋₃ alkenyl or C₂₋₃ alkynyl group;
R⁶ represents a hydrogen atom or a C₁₋₆ alkyl, C₂₋₆ alkenyl, C₂₋₆ alkynyl, amino, C₁₋₆ alkylamino, di(C₁₋₆) alkylamino or C₂₋₇ acylamino group;
each of R⁷ and R⁸ independently represents a hydrogen or halogen atom or a hydroxy, trifluoromethyl, C₁₋₆ alkyl, C₂₋₆ alkenyl, C₂₋₆ alkynyl, C₂₋₇ acyl, C₁₋₆ alkylthio, C₁₋₆ alkoxy or C₃₋₆ cycloalkyl group;
R⁹ represents a hydrogen or halogen atom or a hydroxy, trifluoromethyl, C₁₋₆ alkyl, C₂₋₆ alkenyl, C₂₋₆ alkynyl, C₂₋₇ acyl, C₁₋₆ alkylthio, C₁₋₆ alkoxy or C₃₋₆ cycloalkyl group;
or a salt thereof;
for use in the prevention or treatment of a disease in which raising the intracellular concentration of cAMP is desirable.

28. A compound of general formula I: 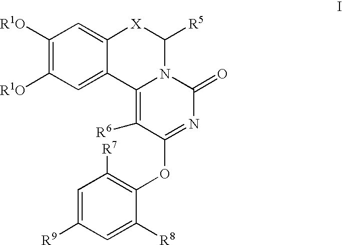
wherein
each of R¹and R² independently represents a C₁₋₆ alkyl or C₂₋₇ acyl group;
X represents OCH₂ or a group CR³R⁴, wherein each of R³ and R⁴ independently represents a hydrogen atom or a C₁₋₃ alkyl group;
R⁵ represents a hydrogen atom or a C₁₋₃ alkyl, C₂₋₃ alkenyl or C₂₋₃ alkynyl group;
R⁶ represents a hydrogen atom or a C₁₋₆ alkyl, C₂₋₆ alkenyl, C₂₋₆ alkynyl, amino, C₁₋₆ alkylamino, di(C₁₋₆) alkylamino or C₂₋₇ acylamino group;
each of R⁷ and R⁸ independently represents a hydrogen or halogen atom or a hydroxy, trifluoromethyl, C₁₋₆ alkyl, C₂₋₆ alkenyl, C₂₋₆ alkynyl, C₂₋₇ acyl, C₁₋₆ alkylthio, C₁₋₆ alkoxy or C₃₋₆ cycloalkyl group;
R⁹ represents a hydrogen or halogen atom or a hydroxy, trifluoromethyl, C₁₋₆ alkyl, C₂₋₆ alkenyl, C₂₋₆ alkynyl, C₂₋₇ acyl, C₁₋₆ alkylthio, C₁₋₆ alkoxy or C₃₋₆ cycloalkyl group;
or a salt thereof;
for use in the prevention or treatment of asthma.

29. A compound of general formula I: 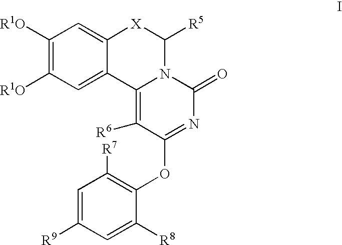
wherein
each of R¹and R² independently represents a C₁₋₆ alkyl or C₂₋₇ acyl group;
X represents OCH₂ or a group CR³R⁴, wherein each of R³ and R⁴ independently represents a hydrogen atom or a C₁₋₃ alkyl group;
R⁵ represents a hydrogen atom or a C₁₋₃ alkyl, C₂₋₃ alkenyl or C₂₋₃ alkynyl group;
R⁶ represents a hydrogen atom or a C₁₋₆ alkyl, C₂₋₆ alkenyl, C₂₋₆ alkynyl, amino, C₁₋₆ alkylamino, di(C₁₋₆) alkylamino or C₂₋₇ acylamino group;
each of R⁷ and R⁸ independently represents a hydrogen or halogen atom or a hydroxy, trifluoromethyl, C₁₋₆ alkyl, C₂₋₆ alkenyl, C₂₋₆ alkynyl, C₂₋₇ acyl, C₁₋₆ alkylthio, C₁₋₆ alkoxy or C₃₋₆ cycloalkyl group;
R⁹ represents a hydrogen or halogen atom or a hydroxy, trifluoromethyl, C₁₋₆ alkyl, C₂₋₆ alkenyl, C₂₋₆ alkynyl, C₂₋₇ acyl, C₁₋₁₆ alkylthio, C₁₋₆ alkoxy or C₃₋₆ cycloalkyl group;
or a salt thereof;
for use in the prevention or treatment of chronic obstructive pulmonary disease (COPD).

30. A compound as claimed in any of claims 25 to 29 wherein the compound is as defined in any of claims 1 to 15.

31. The use of a compound of general formula I: 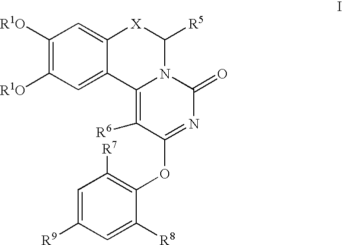
wherein
each of R¹and R² independently represents a C₁₋₆ alkyl or C₂₋₇ acyl group;
X represents OCH₂ or a group CR³R⁴, wherein each of R³ and R⁴ independently represents a hydrogen atom or a C₁₋₃ alkyl group;
R⁵ represents a hydrogen atom or a C₁₋₃ alkyl, C₂₋₃ alkenyl or C₂₋₃ alkyl group;
R⁶ represents a hydrogen atom or a C₁₋₆ alkyl, C₂₋₆ alkenyl, C₂₋₆ alkynyl, amino, C₁₋₆ alkylamino, di(C₁₋₆) alkylamino or C₂₋₇ acylamino group;
each of R⁷ and R⁸ independently represents a hydrogen or halogen atom or a hydroxy, trifluoromethyl, C₁₋₆ alkyl, C₂₋₆ alkenyl, C₂₋₆ alkynyl, C₂₋₇ acyl, C₁₋₆ alkylthio, C₁₋₆ alkoxy or C₃₋₆ cycloalkyl group;
R⁹ represents a hydrogen or halogen atom or a hydroxy, trifluoromethyl, C₁₋₆ alkyl, C₂₋₆ alkenyl, C₂₋₆ alkynyl, C₂₋₇ acyl, C₁₋₆ alkylthio, C₁₋₆ alkoxy or C₃₋₆ cycloalkyl group;
or a salt thereof;
in the manufacture of an inhibitor of a type III/IV phosphodiesterase isoenzyme.

32. The use of a compound of general formula I: 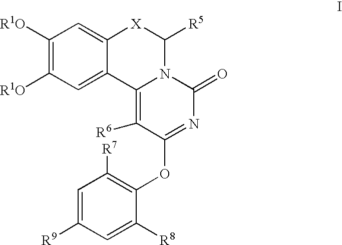
wherein
each of R¹and R² independently represents a C₁₋₆ alkyl or C₂₋₇ acyl group;
X represents OCH₂ or a group CR³R⁴, wherein each of R³ and R⁴ independently represents a hydrogen atom or a C₁₋₃ alkyl group;
R⁵ represents a hydrogen atom or a C₁₋₃ alkyl, C₂₋₃ alkenyl or C₂₋₃ alkynyl group;
R⁶ represents a hydrogen atom or a C₁₋₆ alkyl, C₂₋₆ alkenyl, C₂₋₆ alkynyl, amino, C₁₋₆ alkylamino, di(C₁₋₆) alkylamino or C₂₋₇ acylamino group;
each of R⁷ and R⁸ independently represents a hydrogen or halogen atom or a hydroxy, trifluoromethyl, C₁₋₆ alkyl, C₂₋₆ alkenyl, C₂₋₆ alkynyl, C₂₋₇ acyl, C₁₋₆ alkylthio, C₁₋₄ alkoxy or C₁₋₆ cycloalkyl group;
R⁹ represents a hydrogen or halogen atom or a hydroxy, trifluoromethyl, C₁₋₆ alkyl C₂₋₆ alkenyl, C₂₋₆ alkynyl, C₂₋₇ acyl, C₁₋₆ alkylthio, C₁₋₆ alkoxy or C₃₋₆ cycloalkyl group;
or a salt thereof;
in the manufacture of a bronchodilator.

33. The use of a compound of general formula I: 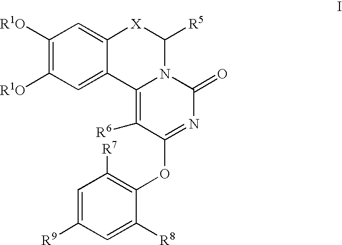
wherein
each of R¹and R² independently represents a C₁₋₆ alkyl or C₂₋₇ acyl group;
X represents OCH₂ or a group CR³R⁴, wherein each of R³ and R⁴ independently represents a hydrogen atom or a C₁₋₃ alkyl group;
R⁵ represents a hydrogen atom or a C₁₋₃ alkyl, C₂₋₃ alkenyl or C₂₋₃ alkynyl group;
R⁶ represents a hydrogen atom or a C₁₋₆ alkyl, C₂₋₆ alkenyl, C₂₋₆ alkynyl, amino, C₁₋₆ alkylamino, di(C₁₋₆) alkylamino or C₂₋₇ acylamino group;
each of R⁷ and R⁸ independently represents a hydrogen or halogen atom or a hydroxy, trifluoromethyl, C₁₋₆ alkyl, C₂₋₆ alkenyl, C₂₋₆ alkynyl, C₂₋₇ acyl, C₁₋₆ alkylthio, C₁₋₆ alkoxy or C₃₋₆ cycloalkyl group;
R⁹ represents a hydrogen or halogen atom or a hydroxy, trifluoromethyl, C₁₋₆ alkyl, C₂₋₆ alkenyl, C₂₋₆ alkynyl, C₂₋₇ acyl, C_{1,6} alkylthio, C₁₋₆ alkoxy or C₃₋₆ cycloalkyl group;
or a salt thereof;
in the manufacture of an anti-asthmatic.

34. The use of a compound of general formula I: 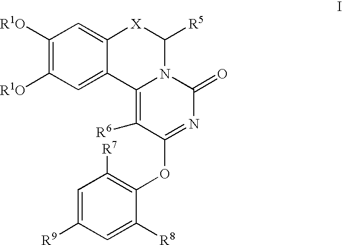
wherein
each of R¹and R² independently represents a C₁₋₆ alkyl or C₂₋₇ acyl group;
X represents OCH₂ or a group CR³R⁴, wherein each of R³ and R⁴ independently represents a hydrogen atom or a C₁₋₃ alkyl group;
R⁵ represents a hydrogen atom or a C₁₋₃ alkyl, C₂₋₃ alkenyl or C₂₋₃ alkynyl group;
R⁶ represents a hydrogen atom or a C₁₋₆ alkyl, C₂₋₆ alkenyl, C₂₋₆ alkynyl, amino, C₁₋₆ alkylamino, di(C₁₋₆) alkylamino or C₂₋₇ acylamino group;
each of R⁷ and R⁸ independently represents a hydrogen or halogen atom or a hydroxy, trifluoromethyl, C₁₋₆ alkyl, C₂₋₆ alkenyl, C₂₋₆ alkynyl, C_{2,7} acyl, C₁₋₆ alkylthio, C₁₋₆ alkoxy or C₃₋₆ cycloalkyl group;
R⁹ represents a hydrogen or halogen atom or a hydroxy, trifluoromethyl, C₁₋₄ alkyl, C₂₋₆ alkenyl, C₂₋₆ alkynyl, C₂₋₇ acyl, C₁₋₆ alkylthio, C₁₋₆ alkoxy or C₃₋₆ cycloalkyl group;
or a salt thereof;
in the manufacture of a medicament for the prevention or treatment of chronic obstructive pulmonary disease (COPD).

35. The use as claimed in any of claims 31 to 34, wherein the compound is as defined in any of claims 1 to 15.

36. A method for the treatment or prevention of a disease in a mammal where a phosphodiesterase isoenzyme inhibitor and/or a bronchodilator would be expected to be of benefit, which method comprises administering to said mammal an effective, non-toxic amount of a compound of general formula I: 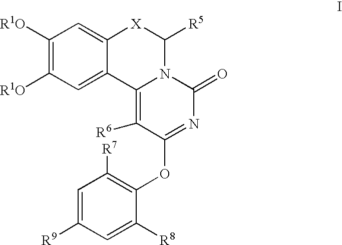
wherein
each of R¹ and R² independently represents a C₁₋₆ alkyl or C₂₋₇ acyl group;
X represents OCH₂ or a group CR³R⁴, wherein each of R³ and R⁴ independently represents a hydrogen atom or a C₁₋₃ alkyl group;
R⁵ represents a hydrogen atom or a C₁₋₃ alkyl, C₂₋₃ alkenyl or C₂₋₃ alkynyl group;
R⁶ represents a hydrogen atom or a C₁₋₆ alkyl, C₂₋₆ alkenyl, C₂₋₆ alkynyl, amino, C₁₋₆ alkylamino, di(C₁₋₆) alkylamino or C₂₋₇ acylamino group;
each of R⁷ and R⁸ independently represents a hydrogen or halogen atom or a hydroxy, trifluoromethyl, C₁₋₆ alkyl, C₂₋₆ alkenyl, C₂₋₆ alkynyl, C₂₋₇ acyl, C₁₋₆ alkylthio, C₁₋₆ alkoxy or C₃₋₆ cycloalkyl group;
R⁹ represents a hydrogen or halogen atom or a hydroxy, trifluoromethyl, C_{1,6} alkyl, C₂₋₆ alkenyl, C₂₋₆ alkynyl, C₂₋₇ acyl, C₁₋₆ alkylthio, C₁₋₆ alkoxy or C₃₋₆ cycloalkyl group;
or a salt thereof.

37. A method for the treatment or prevention of asthma in a mammal, which method comprises administering to said mammal an effective, non-toxic amount of a compound of general formula I: 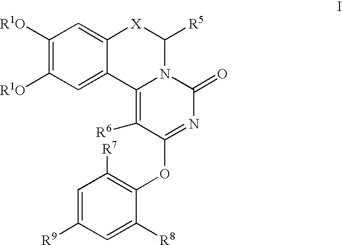
wherein
each of R¹ and R² independently represents a C₁₋₆ alkyl or C₂₋₇ acyl group;
X represents OCH₂ or a group CR³R⁴, wherein each of R³ and R⁴ independently represents a hydrogen atom or a C₁₋₃ alkyl group;
R⁵ represents a hydrogen atom or a C₁₋₃ alkyl, C₂₋₃ alkenyl or C₂₋₃ alkynyl group;
R⁶ represents a hydrogen atom or a C₁₋₆ alkyl, C₂₋₆ alkenyl, C₂₋₆ alkynyl, amino, C₁₋₆ alkylamino, di(C₁₋₆) alkylamino or C₂₋₇ acylamino group;
each of R⁷ and R⁸ independently represents a hydrogen or halogen atom or a hydroxy, trifluoromethyl, C₁₋₆ alkyl, C₂₋₆ alkenyl, C₂₋₆ alkynyl, C₂₋₇ acyl, C₁₋₆ alkylthio, C₁₋₆ alkoxy or C₃₋₆ cycloalkyl group;
R⁹ represents a hydrogen or halogen atom or a hydroxy, trifluoromethyl, C₁₋₆ alkyl, C₂₋₆ alkenyl, C₂₋₆ alkynyl, C₂₋₇ acyl, C₁₋₆ alkylthio, C₁₋₆ alkoxy or C₃₋₆ cycloalkyl group;
or a salt thereof.

38. A method for the treatment or prevention of chronic obstructive pulmonary disease (COPD) in a mammal, which method comprises administering to said mammal an effective, non-toxic amount of a compound of general formula I: 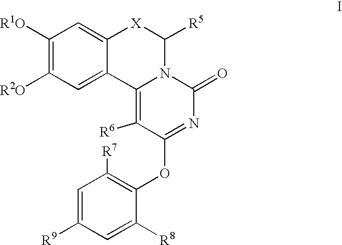
wherein
each of R¹ and R² independently represents a C₁₋₆ alkyl or C₂₋₇ acyl group;
X represents OCH₂ or a group CR³R⁴, wherein each of R³ and R⁴ independently represents a hydrogen atom or a C₁₋₃ alkyl group;
R⁵ represents a hydrogen atom or a C₁₋₃ alkyl, C₂₋₃ alkenyl or C₂₋₃ alkynyl group;
R⁶ represents a hydrogen atom or a C₁₋₆ alkyl, C₂₋₆ alkenyl, C₂₋₆ alkynyl, amino, C₁₋₆ alkylamino, di(C₁₋₆) alkylamino or C₂₋₇ acylamino group;
each of R⁷ and R⁸ independently represents a hydrogen or halogen atom or a hydroxy, trifluoromethyl, C₁₋₆ alkyl, C₂₋₆ alkenyl, C₂₋₆ alkynyl, C₂₋₇ acyl, C₁₋₆ alkylthio, C₁₋₆ alkoxy or C₃₋₆ cycloalkyl group;
R⁹ represents a hydrogen or halogen atom or a hydroxy, trifluoromethyl, C₁₋₆ alkyl, C₂₋₆ alkenyl, C₂₋₆ alkynyl, C₂₋₇ acyl, C₁₋₆ alkylthio, C₁₋₆ alkoxy or C₃₋₆ cycloalkyl group;
or a salt thereof.

39. A method as claimed in claim 36, 37 or 38, wherein the compound is as defined in any of claims 1 to 15.

40. A method as claimed in any of claims 36 to 39, wherein the compound is administered by aerosol.

41. A method as claimed in any of claims 36 to 40, wherein the animal is a human.

42. A compound substantially as hereinbefore described in any of the examples.

43. A process substantially as hereinbefore described in any of the examples.
